(19) United States
(12) Patent Application Publication
(10) Pub. No.: US 2024/0112751 A1
(43) Pub. Date: Apr. 4, 2024

(54) COPY NUMBER VARIATION (CNV) BREAKPOINT DETECTION

(71) Applicant: Illumina, Inc., San Diego, CA (US)

(72) Inventors: Anindita Dutta, San Francisco, CA (US); Elahe Aghapour, San Mateo, CA (US)

(21) Appl. No.: 18/477,346

(22) Filed: Sep. 28, 2023

Related U.S. Application Data
(60) Provisional application No. 63/411,581, filed on Sep. 29, 2022.

(51) Int. Cl. G16B 20/10; G16B 40/00

(52) U.S. Cl. CPC G16B 20/10; G16B 40/00

(57) ABSTRACT

A method of processing sequence data comprising a known location of the start of a copy number variant breakpoint to generate a prediction for the location of the end of the copy number variant breakpoint. The method comprises an encoder and a copy number variation (CNV) caller guide. The encoder processes an anchor sequence and corresponding subject candidate sequence to generate a learned representation of the anchor sequence and a learned representation of the corresponding subject candidate sequence. The CNV caller guide determines a similarity between the learned representation of the anchor sequence and a learned representation of the corresponding subject candidate sequence. Similarity between anchor sequence and subject candidate sequence is used as a proxy for likelihood that the end of the CNV breakpoint is located on the subject candidate sequence.

### CROSS-REFERENCE TO RELATED APPLICATIONS

The present application claims the benefit of, and priority to, U.S. Provisional Application No. 63/411,581, entitled “COPY NUMBER VARIATION (CNV) BREAKPOINT DETECTION,” filed on Sep. 29, 2022. The aforementioned application is hereby incorporated by reference in its entirety.

### FIELD OF THE TECHNOLOGY DISCLOSED

The technology disclosed relates to artificial intelligence type computers and digital data processing systems and corresponding data processing methods and products for emulation of intelligence (i.e., knowledge based systems, reasoning systems, and knowledge acquisition systems); and including systems for reasoning with uncertainty (e.g., fuzzy logic systems), adaptive systems, machine learning systems, and artificial neural networks. In particular, the technology disclosed relates to using deep convolutional neural networks to analyze data.

### INCORPORATIONS BY REFERENCE

The following are incorporated by reference for all purposes as if fully set forth herein:
Sundaram, L. et al. Predicting the clinical impact of human mutation with deep neural networks. Nat. Genet. 50, 1161-1170 (2018);
Jaganathan, K. et al. Predicting splicing from primary sequence with deep learning. Cell 176, 535-548 (2019);
U.S. Patent Application No. 62/573,144, titled “TRAINING A DEEP PATHOGENICITY CLASSIFIER USING LARGE-SCALE BENIGN TRAINING DATA,” filed Oct. 16, 2017 (Attorney Docket No. ILLM 1000-1/IP-1611-PRV);
U.S. Patent Application No. 62/573,149, titled “PATHOGENICITY CLASSIFIER BASED ON DEEP CONVOLUTIONAL NEURAL NETWORKS (CNNs),” filed Oct. 16, 2017 (Attorney Docket No. ILLM 1000-2/IP-1612-PRV);
U.S. Patent Application No. 62/573,153, titled “DEEP SEMI-SUPERVISED LEARNING THAT GENERATES LARGE-SCALE PATHOGENIC TRAINING DATA,” filed Oct. 16, 2017 (Attorney Docket No. ILLM 1000-3/IP-1613-PRV);
U.S. Patent Application No. 62/582,898, titled “PATHOGENICITY CLASSIFICATION OF GENOMIC DATA USING DEEP CONVOLUTIONAL NEURAL NETWORKS (CNNs),” filed Nov. 7, 2017 (Attorney Docket No. ILLM 1000-4/IP-1618-PRV);
U.S. patent application Ser. No. 16/160,903, titled “DEEP LEARNING-BASED TECHNIQUES FOR TRAINING DEEP CONVOLUTIONAL NEURAL NETWORKS,” filed on Oct. 15, 2018 (Attorney Docket No. ILLM 1000-5/IP-1611-US);
U.S. patent application Ser. No. 16/160,986, titled “DEEP CONVOLUTIONAL NEURAL NETWORKS FOR VARIANT CLASSIFICATION,” filed on Oct. 15, 2018 (Attorney Docket No. ILLM 1000-6/IP-1612-US);
U.S. patent application Ser. No. 16/160,968, titled “SEMI-SUPERVISED LEARNING FOR TRAINING AN ENSEMBLE OF DEEP CONVOLUTIONAL NEURAL NETWORKS,” filed on Oct. 15, 2018 (Attorney Docket No. ILLM 1000-7/IP-1613-US);
U.S. patent application Ser. No. 16/160,978, titled “DEEP LEARNING-BASED SPLICE SITE CLASSIFICATION,” filed on Oct. 15, 2018 (Attorney Docket No. ILLM 1001-4/IP-1680-US);
U.S. patent application Ser. No. 16/407,149, titled “DEEP LEARNING-BASED TECHNIQUES FOR PRE-TRAINING DEEP CONVOLUTIONAL NEURAL NETWORKS,” filed May 8, 2019 (Attorney Docket No. ILLM 1010-1/IP-1734-US);
U.S. patent application Ser. No. 17/232,056, titled “DEEP CONVOLUTIONAL NEURAL NETWORKS TO PREDICT VARIANT PATHOGENICITY USING THREE-DIMENSIONAL (3D) PROTEIN STRUCTURES,” filed on Apr. 15, 2021, (Atty. Docket No. ILLM 1037-2/IP-2051-US);
U.S. Patent Application No. 63/175,495, titled “MULTI-CHANNEL PROTEIN VOXELIZATION TO PREDICT VARIANT PATHOGENICITY USING DEEP CONVOLUTIONAL NEURAL NETWORKS,” filed on Apr. 15, 2021, (Atty. Docket No. ILLM 1047-1/IP-2142-PRV);
U.S. Patent Application No. 63/175,767, titled “EFFICIENT VOXELIZATION FOR DEEP LEARNING,” filed on Apr. 16, 2021, (Atty. Docket No. ILLM 1048-1/IP-2143-PRV);
U.S. patent application Ser. No. 17/468,411, titled “ARTIFICIAL INTELLIGENCE-BASED ANALYSIS OF PROTEIN THREE-DIMENSIONAL (3D) STRUCTURES,” filed on Sep. 7, 2021, (Atty. Docket No. ILLM 1037-3/IP-2051A-US);
U.S. Provisional Patent Application No. 63/253,122, titled “PROTEIN STRUCTURE-BASED PROTEIN LANGUAGE MODELS,” filed Oct. 6, 2021 (Attorney Docket No. ILLM 1050-1/IP-2164-PRV);
U.S. Provisional Patent Application No. 63/281,579, titled “PREDICTING VARIANT PATHOGENICITY FROM EVOLUTIONARY CONSERVATION USING THREE-DIMENSIONAL (3D) PROTEIN STRUCTURE VOXELS,” filed Nov. 19, 2021 (Attorney Docket No. ILLM 1060-1/IP-2270-PRV);
U.S. Provisional Patent Application No. 63/281,592, titled “COMBINED AND TRANSFER LEARNING OF A VARIANT PATHOGENICITY PREDICTOR USING GAPED AND NON-GAPED PROTEIN SAMPLES,” filed Nov. 19, 2021 (Attorney Docket No. ILLM 1061-1/IP-2271-PRV);
U.S. Provisional Patent Application No. 63/294,813, titled “PERIODIC MASK PATTERN FOR REVELATION LANGUAGE MODELS,” filed Dec. 29, 2021 (Attorney Docket No. ILLM 1063-1/IP-2296-PRV);
U.S. Provisional Patent Application No. 63/294,816, titled “CLASSIFYING MILLIONS OF VARIANTS OF UNCERTAIN SIGNIFICANCE USING PRIMATE SEQUENCING AND DEEP LEARNING,” filed Dec. 29, 2021 (Attorney Docket No. ILLM 1064-1/IP-2297-PRV);
U.S. Provisional Patent Application No. 63/294,820, titled “IDENTIFYING GENES WITH DIFFERENTIAL SELECTIVE CONSTRAINT BETWEEN HUMANS AND NON-HUMAN PRIMATES,” filed Dec. 29, 2021 (Attorney Docket No. ILLM 1065-1/IP-2298-PRV);
U.S. Provisional Patent Application No. 63/294,827, titled “DEEP LEARNING NETWORK FOR EVOLUTIONARY CONSERVATION,” filed Dec. 29, 2021 (Attorney Docket No. ILLM 1066-1/IP-2299-PRV);
U.S. Provisional Patent Application No. 63/294,828, titled “INTER-MODEL PREDICTION SCORE RECALIBRATION,” filed Dec. 29, 2021 (Attorney Docket No. ILLM 1067-1/IP-2301-PRV); and
U.S. Provisional Patent Application No. 63/294,830, titled “SPECIES-DIFFERENTIABLE EVOLUTIONARY PROFILES,” filed Dec. 29, 2021 (Attorney Docket No. ILLM 1068-1/IP-2302-PRV).

### BACKGROUND

The subject matter discussed in this section should not be assumed to be prior art merely as a result of its mention in this section. Similarly, a problem mentioned in this section or associated with the subject matter provided as background should not be assumed to have been previously recognized in the prior art. The subject matter in this section merely represents different approaches, which in and of themselves can also correspond to implementations of the claimed technology.

Genomics, in the broad sense, also referred to as functional genomics, aims to characterize the function of every genomic element of an organism by using genome-scale assays such as genome sequencing, transcriptome profiling, and proteomics. Genomics arose as a data-driven science—it operates by discovering novel properties from explorations of genome-scale data rather than by testing preconceived models and hypotheses. Applications of genomics include finding associations between genotype and phenotype, discovering biomarkers for patient stratification, predicting the function of genes, and charting biochemically active genomic regions and residues such as transcriptional enhancers and single nucleotide polymorphisms (SNPs).

Genomics data are too large and too complex to be mined solely by visual investigation of pairwise correlations. Instead, analytical tools are required to support the discovery of unanticipated relationships, to derive novel hypotheses and models, and to make predictions. Unlike some algorithms, in which assumptions and domain expertise are hard coded, machine learning algorithms are designed to automatically detect patterns in data. Hence, machine learning algorithms are suited to data-driven sciences and, in particular, to genomics. However, the performance of machine learning algorithms can strongly depend on how the data are represented, that is, on how each variable (also called a feature) is computed. For instance, to classify a tumor as malign or benign from a fluorescent microscopy image, a preprocessing algorithm could detect cells, identify the cell type, and generate a list of cell counts for each cell type.

A machine learning model can take the estimated cell counts, which are examples of handcrafted features, as input features to classify the tumor. A central issue is that classification performance depends heavily on the quality and the relevance of these features. For example, relevant visual features such as cell morphology, distances between cells, or localization within an organ are not captured in cell counts, and this incomplete representation of the data may reduce classification accuracy.

An opportunity arises to develop a machine learning model that can be applied to copy number variation (CNV) as it relates to human diseases such a schizophrenia. The technology disclosed below implements a targeted CNV caller guide applied to identify regions of the reference genome where CNVs are likely and to serve as a guide to targeted CNV callers. Accurate distinction of genomic CNVs may result in benefit for both precision medicine and targeted drug development.

### BRIEF DESCRIPTION OF THE DRAWINGS

In the drawings, like reference characters generally refer to like parts throughout the different views. Also, the drawings are not necessarily to scale, with an emphasis instead generally being placed upon illustrating the principles of the technology disclosed. In the following description, various implementations of the technology disclosed are described with reference to the following drawings, in which.

FIG. 1A is a schematic diagram illustrating a system for detecting the end of a CNV breakpoint.

FIG. 1B is a schematic diagram illustrating a system for detecting the start of a CNV breakpoint.

FIG. 1C is a schematic diagram illustrating a system for detecting the start and end of a CNV breakpoint.

FIG. 2A is a schematic diagram of an autoencoder for sequence compression and sequence reconstruction.

FIG. 2B is a schematic diagram of an encoder for sequence compression.

FIG. 3 illustrates the vectorization of a reference genomic sequence.

FIG. 4A is a schematic diagram illustrating a system for harvesting augmented sequences from a real reference sequence.

FIG. 4B illustrates a plurality of harvested sequences from a real reference sequence.

FIG. 5 is an illustration of WaveNet architecture.

FIG. 6A is a schematic diagram of a triplet loss model.

FIG. 6B shows an equation for triplet loss functions.

FIG. 7 is a schematic diagram of a Siamese CNV caller guide.

FIG. 8 illustrates examples of genomic variation.

FIG. 9 illustrates an example CNV breakpoint.

FIG. 10 shows performance results of the disclosed encoder.

FIGS. 11A-11D show performance results of the disclosed CNV caller guide.

FIG. 12 shows an example computer system that can be used to implement the technology disclosed.

### DETAILED DESCRIPTION

The following discussion is presented to enable any person skilled in the art to make and use the technology disclosed and is provided in the context of a particular application and its requirements. Various modifications to the disclosed implementations will be readily apparent to those skilled in the art, and the general principles defined herein may be applied to other implementations and applications without departing from the spirit and scope of the technology disclosed. Thus, the technology disclosed is not intended to be limited to the implementations shown but is to be accorded the widest scope consistent with the principles and features disclosed herein.

The detailed description of various implementations will be better understood when read in conjunction with the appended drawings. To the extent that the figures illustrate diagrams of the functional blocks of the various implementations, the functional blocks are not necessarily indicative of the division between hardware circuitry. Thus, for example, one or more of the functional blocks (e.g., modules, processors, or memories) may be implemented in a single piece of hardware (e.g., a general purpose signal processor or a block of random access memory, hard disk, or the like) or multiple pieces of hardware. Similarly, the programs may be stand-alone programs, may be incorporated as subroutines in an operating system, may be functions in an installed software package, and the like. It should be understood that the various implementations are not limited to the arrangements and instrumentality shown in the drawings.

The processing engines and databases of the figures, designated as modules, can be implemented in hardware or software, and need not be divided up in precisely the same blocks as shown in the figures. Some of the modules can also be implemented on different processors, computers, or servers, or spread among a number of different processors, computers, or servers. In addition, it will be appreciated that some of the modules can be combined, operated in parallel or in a different sequence than that shown in the figures without affecting the functions achieved. The modules in the figures can also be thought of as flowchart steps in a method. A module also need not necessarily have all its code disposed contiguously in memory; some parts of the code can be separated from other parts of the code with code from other modules or other functions disposed in between.

### Introduction

The technology disclosed addresses the problem of detecting copy number variation (CNV) breakpoints within a genome. Genetic variation describes the variation within the DNA sequence in individual genomes. CNVs are defined as structural variations in the genome larger than one kilobase (kB) in size and can involve gains or losses of genomic DNA. Evidence is accumulating that CNVs play important roles in many human diseases (e.g., schizophrenia). Current de novo CNV callers have substantial limitations, including redundant detection of the same CNV in each sample, CNV size limitations, and inaccuracy due to the high variability of breakpoint structural patterns. The technology disclosed comprises a targeted CNV caller guide that aims to identify regions of the reference genome where CNVs are likely and serve as a guide to targeted CNV callers. The goal of the targeted CNV caller guide model disclosed is to learn predisposition of regions of the genome to have a CNV by harnessing possible homology between CNV start point regions and CNV end point regions via training the targeted CNV caller guide model on a high-quality CNV ground truth dataset collected across a plurality of samples.

In one implementation of the technology disclosed, the targeted CNV caller guide model may be trained to predict a CNV end point from a CNV start point. In another implementation of the technology disclosed, the targeted CNV caller guide model may be trained to predict a CNV start point from a CNV end point. In yet another implementation of the technology disclosed, the targeted CNV caller guide model may be trained to predict unlabeled CNVs in the reference genome.

The disclosed method processes sequencing data comprising a CNV start point to generate the location of a predicted CNV end point, wherein the CNV end point is downstream of the input CNV start point. The input sequence data comprises an anchor sequence and a plurality of downstream candidate sequences, wherein the CNV start point is located on the anchor sequence and the CNV end point is located on one of the candidate sequences. Candidate sequences are iteratively tested as input for the targeted CNV caller guide model, along with the anchor sequence, to generate an output that generates a prediction of whether the end of the CNV breakpoint is located on the particular subject candidate sequence. In some implementation of the technology disclosed, the above procedure is applied to predicting a CNV start point from the processing of a CNV end point (i.e., such that the CNV end point is located on the anchor sequence and the CNV start point is located on one of a plurality of upstream candidate sequences). In other implementation of the technology disclosed, the above procedure is applied to predicting both CNV breakpoint regions from the processing of a genomic sequence (i.e., such that the model generates an output of two candidate sequences likely to be corresponding CNV breakpoint start and end points).

In some implementations of the trained CNV caller guide model, comprises a trained encoder and a trained multi-layer perceptron. The trained encoder processes the input anchor sequence and subject candidate sequence to generate a compressed representation of each sequence, respectively. Next, the trained multi-layer perceptron determines a similarity (i.e., a distance score) between the anchor sequence and the subject candidate sequence by comparing the compressed representation of the anchor sequence against the compressed representation of the subject candidate sequence. If the distance score is below a certain distance threshold, the trained CNV caller guide model generates an output that specifies that the end of the CNV breakpoint is located on the particular subject candidate sequence. Note that this Application uses the terms “CNV start point” and “start of the CNV breakpoint” synonymously, as well as using the terms “CNV end point” and “end of the CNV breakpoint” synonymously.

### Application of Deep Learning to CNV, Generally

Deep learning, a subdiscipline of machine learning, addresses this issue by embedding the computation of features into the machine learning model itself to yield end-to-end models. This outcome has been realized through the development of deep neural networks, machine learning models that comprise successive elementary operations, which compute increasingly more complex features by taking the results of preceding operations as input. Deep neural networks are able to improve prediction accuracy by discovering relevant features of high complexity, such as the cell morphology and spatial organization of cells in the above example. The construction and training of deep neural networks have been enabled by the explosion of data, algorithmic advances, and substantial increases in computational capacity, particularly through the use of graphical processing units (GPUs).

The goal of supervised learning is to obtain a model that takes features as input and returns a prediction for a so-called target variable. An example of a supervised learning problem is one that predicts whether an intron is spliced out or not (the target) given features on the RNA such as the presence or absence of the canonical splice site sequence, and the location of the splicing branchpoint or intron length. Training a machine learning model refers to learning its parameters, which commonly involves minimizing a loss function on training data with the aim of making accurate predictions on unseen data.

For many supervised learning problems in computational biology, the input data can be represented as a table with multiple columns, or features, each of which contains numerical or categorical data that are potentially useful for making predictions. Some input data are naturally represented as features in a table (such as temperature or time), whereas other input data need to be first transformed (such as deoxyribonucleic acid (DNA) sequence into k-mer counts) using a process called feature extraction to fit a tabular representation. For the intron-splicing prediction problem, the presence or absence of the canonical splice site sequence, the location of the splicing branchpoint and the intron length can be preprocessed features collected in a tabular format. Tabular data are standard for a wide range of supervised machine learning models, ranging from simple linear models, such as logistic regression, to more flexible nonlinear models, such as neural networks, and many others.

Logistic regression is a binary classifier, that is, a supervised learning model that predicts a binary target variable. Specifically, logistic regression predicts the probability of the positive class by computing a weighted sum of the input features mapped to the [0,1] interval using the sigmoid function, a type of activation function. The parameters of logistic regression, or other linear classifiers that use different activation functions, are the weights in the weighted sum. Linear classifiers fail when the classes, for instance, that of an intron spliced out or not, cannot be well discriminated with a weighted sum of input features. To improve predictive performance, new input features can be manually added by transforming or combining existing features in new ways, for example, by taking powers or pairwise products.

Neural networks use hidden layers to learn these nonlinear feature transformations automatically. Each hidden layer can be thought of as multiple linear models with their output transformed by a nonlinear activation function, such as the sigmoid function or the more popular rectified-linear unit (ReLU). Together, these layers compose the input features into relevant complex patterns, which facilitates the task of distinguishing two classes.

Deep neural networks use many hidden layers, and a layer is said to be fully-connected when each neuron receives inputs from all neurons of the preceding layer. Neural networks are commonly trained using stochastic gradient descent, an algorithm suited to training models on very large data sets. Implementation of neural networks using modern deep learning frameworks enables rapid prototyping with different architectures and data sets. Fully-connected neural networks can be used for a number of genomics applications, which include predicting the percentage of exons spliced in for a given sequence from sequence features such as the presence of binding motifs of splice factors or sequence conservation; prioritizing potential disease-causing genetic variants; and predicting cis-regulatory elements in a given genomic region using features such as chromatin marks, gene expression and evolutionary conservation.

Local dependencies in spatial and longitudinal data must be considered for effective predictions. For example, shuffling a DNA sequence or the pixels of an image severely disrupts informative patterns. These local dependencies set spatial or longitudinal data apart from tabular data, for which the ordering of the features is arbitrary. Consider the problem of classifying genomic regions as bound versus unbound by a particular transcription factor, in which bound regions are defined as high-confidence binding events in chromatin immunoprecipitation followed by sequencing (ChIP-seq) data. Transcription factors bind to DNA by recognizing sequence motifs. A fully-connected layer based on sequence-derived features, such as the number of k-mer instances or the position weight matrix (PWM) matches in the sequence, can be used for this task. As k-mer or PWM instance frequencies are robust to shifting motifs within the sequence, such models could generalize well to sequences with the same motifs located at different positions. However, they would fail to recognize patterns in which transcription factor binding depends on a combination of multiple motifs with well-defined spacing. Furthermore, the number of possible k-mers increases exponentially with k-mer length, which poses both storage and overfitting challenges.

A convolutional layer is a special form of fully-connected layer in which the same fully-connected layer is applied locally, for example, in a 6 bp window, to all sequence positions. This approach can also be viewed as scanning the sequence using multiple PWMs, for example, for transcription factors GATA1 and TAL1. By using the same model parameters across positions, the total number of parameters is drastically reduced, and the network is able to detect a motif at positions not seen during training. Each convolutional layer scans the sequence with several filters by producing a scalar value at every position, which quantifies the match between the filter and the sequence. As in fully-connected neural networks, a nonlinear activation function (commonly ReLU) is applied at each layer. Next, a pooling operation is applied, which aggregates the activations in contiguous bins across the positional axis, commonly taking the maximal or average activation for each channel. Pooling reduces the effective sequence length and coarsens the signal. The subsequent convolutional layer composes the output of the previous layer and is able to detect whether a GATA1 motif and TAL1 motif were present at some distance range. Finally, the output of the convolutional layers can be used as input to a fully-connected neural network to perform the final prediction task. Hence, different types of neural network layers (e.g., fully-connected layers and convolutional layers) can be combined within a single neural network.

Convolutional neural networks (CNNs) can predict various molecular phenotypes on the basis of DNA sequence alone. Applications include classifying transcription factor binding sites and predicting molecular phenotypes such as chromatin features, DNA contact maps, DNA methylation, gene expression, translation efficiency, RBP binding, and microRNA (miRNA) targets. In addition to predicting molecular phenotypes from the sequence, convolutional neural networks can be applied to more technical tasks traditionally addressed by handcrafted bioinformatics pipelines. For example, convolutional neural networks can predict the specificity of guide RNA, denoise ChIP-seq, enhance Hi-C data resolution, predict the laboratory of origin from DNA sequences and call genetic variants. Convolutional neural networks have also been employed to model long-range dependencies in the genome. Although interacting regulatory elements may be distantly located on the unfolded linear DNA sequence, these elements are often proximal in the actual 3D chromatin conformation. Hence, modelling molecular phenotypes from the linear DNA sequence, albeit a crude approximation of the chromatin, can be improved by allowing for long-range dependencies and allowing the model to implicitly learn aspects of the 3D organization, such as promoter-enhancer looping. This is achieved by using dilated convolutions, which have a receptive field of up to 32 kb. Dilated convolutions also allow splice sites to be predicted from sequence using a receptive field of 10 kb, thereby enabling the integration of genetic sequences across distances as long as typical human introns (See Jaganathan, K. et al. Predicting splicing from primary sequence with deep learning. Cell 176, 535-548 (2019)).

Different types of neural networks can be characterized by their parameter-sharing schemes. For example, fully-connected layers have no parameter sharing, whereas convolutional layers impose translational invariance by applying the same filters at every position of their input. Recurrent neural networks (RNNs) are an alternative to convolutional neural networks for processing sequential data, such as DNA sequences or time series, that implement a different parameter-sharing scheme. Recurrent neural networks apply the same operation to each sequence element. The operation takes as input the memory of the previous sequence element and the new input. It updates the memory and optionally emits an output, which is either passed on to subsequent layers or is directly used as model predictions. By applying the same model to each sequence element, recurrent neural networks are invariant to the position index in the processed sequence. For example, a recurrent neural network can detect an open reading frame in a DNA sequence regardless of the position in the sequence. This task requires the recognition of a certain series of inputs, such as the start codon followed by an in-frame stop codon.

The main advantage of recurrent neural networks over convolutional neural networks is that they are, in theory, able to carry over information through infinitely long sequences via memory. Furthermore, recurrent neural networks can naturally process sequences of widely varying length, such as mRNA sequences. However, convolutional neural networks combined with various tricks (such as dilated convolutions) can reach comparable or even better performances than recurrent neural networks on sequence-modelling tasks, such as audio synthesis and machine translation. Recurrent neural networks can aggregate the outputs of convolutional neural networks for predicting single-cell DNA methylation states, RBP binding, transcription factor binding, and DNA accessibility. Moreover, because recurrent neural networks apply a sequential operation, they cannot be easily parallelized and are hence much slower to compute than convolutional neural networks.

Each human has a unique genetic code, though a large portion of the human genetic code is common for all humans. In some cases, a human genetic code may include an outlier, called a genetic variant, that may be common among individuals of a relatively small group of the human population. For example, a particular human protein may comprise a specific sequence of amino acids, whereas a variant of that protein may differ by one amino acid in the otherwise same specific sequence.

Genetic variants may be pathogenetic, leading to diseases. Though most of such genetic variants have been depleted from genomes by natural selection, an ability to identify which genetic variants are likely to be pathogenic can help researchers focus on these genetic variants to gain an understanding of the corresponding diseases and their diagnostics, treatments, or cures. The clinical interpretation of millions of human genetic variants remains unclear. Some of the most frequent pathogenic variants are single nucleotide missense mutations that change the amino acid of a protein. However, not all missense mutations are pathogenic.

Models that can predict molecular phenotypes directly from biological sequences can be used as in silico perturbation tools to probe the associations between genetic variation and phenotypic variation and have emerged as new methods for quantitative trait loci identification and variant prioritization. These approaches are of major importance given that the majority of variants identified by genome-wide association studies of complex phenotypes are non-coding, which makes it challenging to estimate their effects and contribution to phenotypes. Moreover, linkage disequilibrium results in blocks of variants being co-inherited, which creates difficulties in pinpointing individual causal variants. Thus, sequence-based deep learning models that can be used as interrogation tools for assessing the impact of such variants offer a promising approach to finding potential drivers of complex phenotypes. One example includes predicting the effect of non-coding single-nucleotide variants and short insertions or deletions (indels) indirectly from the difference between two variants in terms of transcription factor binding, chromatin accessibility, or gene expression predictions. Another example includes predicting novel splice site creation from sequence or quantitative effects of genetic variants on splicing.

End-to-end deep learning approaches for variant effect predictions are applied to predict the pathogenicity of missense variants from protein sequence and sequence conservation data (See Sundaram, L. et al. Predicting the clinical impact of human mutation with deep neural networks. Nat. Genet. 50, 1161-1170 (2018), referred to herein as “PrimateAI”). PrimateAI uses deep neural networks trained on variants of known pathogenicity with data augmentation using cross-species information. In particular, PrimateAI uses sequences of wild-type and mutant proteins to compare the difference and decide the pathogenicity of mutations using the trained deep neural networks. Such an approach that utilizes the protein sequences for pathogenicity prediction is promising because it can avoid the circularity problem and overfilling to previous knowledge. However, compared to the adequate number of data to train the deep neural networks effectively, the number of clinical data available in ClinVar is relatively small. To overcome this data scarcity, PrimateAI uses common human variants and variants from primates as benign data while simulated variants based on trinucleotide context were used as unlabeled data.

PrimateAI outperforms prior methods when trained directly upon sequence alignments. PrimateAI learns important protein domains, conserved amino acid positions, and sequence dependencies directly from the training data consisting of about 120,000 human samples. PrimateAI substantially exceeds the performance of other variant pathogenicity prediction tools in differentiating benign and pathogenic de-novo mutations in candidate developmental disorder genes, and in reproducing prior knowledge in ClinVar. These results suggest that PrimateAI is an important step forward for variant classification tools that may lessen the reliance of clinical reporting on prior knowledge.

Central to protein biology is the understanding of how structural elements give rise to observed function. The surfeit of protein structural data enables the development of computational methods to systematically derive rules governing structural-functional relationships. However, the performance of these methods depends critically on the choice of protein structural representation.

Protein sites are microenvironments within a protein structure, distinguished by their structural or functional role. A site can be defined by a three-dimensional (3D) location and a local neighborhood around this location in which the structure or function exists. Central to rational protein engineering is the understanding of how the structural arrangement of amino acids creates functional characteristics within protein sites. Determination of the structural and functional roles of individual amino acids within a protein provides information to help engineer and alter protein functions. Identifying functionally or structurally important amino acids allows focused engineering efforts such as site-directed mutagenesis for altering targeted protein functional properties. Alternatively, this knowledge can help avoid engineering designs that would abolish a desired function.

Since it has been established that structure is far more conserved than sequence, the increase in protein structural data provides an opportunity to systematically study the underlying pattern governing the structural-functional relationships using data-driven approaches. A fundamental aspect of any computational protein analysis is how protein structural information is represented. The performance of machine learning methods often depends more on the choice of data representation than the machine learning algorithm employed. Good representations efficiently capture the most critical information while poor representations create a noisy distribution with no underlying patterns.

The surfeit of protein structures and the recent success of deep learning algorithms provide an opportunity to develop tools for automatically extracting task-specific representations of protein structures.

Copy number variation (CNV) is defined as structural variation in the genome larger than one kilobase (kB) in size and can involve gains or losses of genomic DNA. Evidence is accumulating that CNVs play important roles in many human diseases (e.g., schizophrenia). Current de novo CNV callers have substantial limitations, including redundant detection of the same CNV in each sample, CNV size limitations, and inaccuracy due to the high variability of breakpoint structural patterns. An opportunity arises for a targeted CNV caller guide that aims to identify regions of the reference genome where CNVs are likely and serve as a guide to targeted CNV callers. Accurate distinction of genomic CNVs may result in benefit for both precision medicine and targeted drug development.

### System Overview

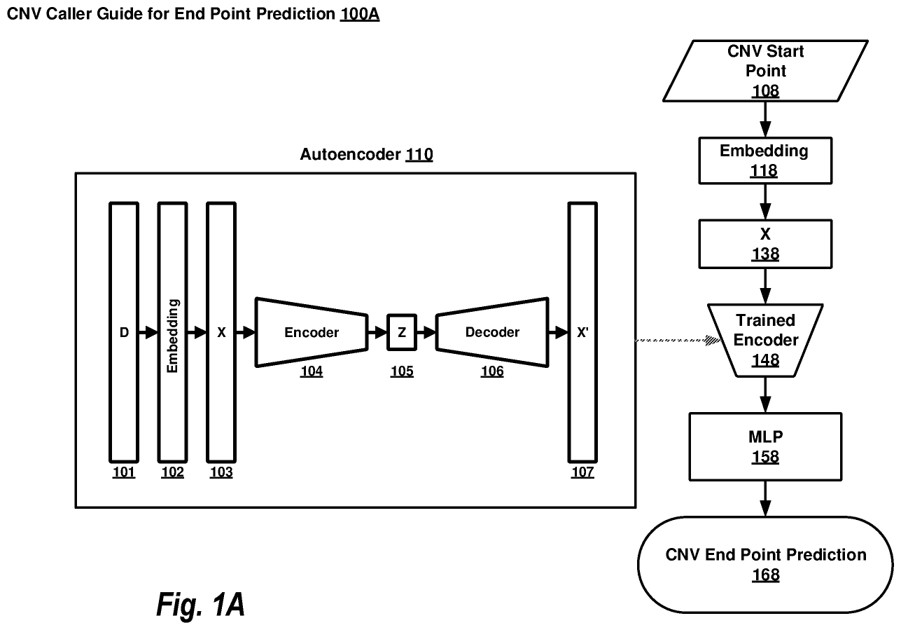

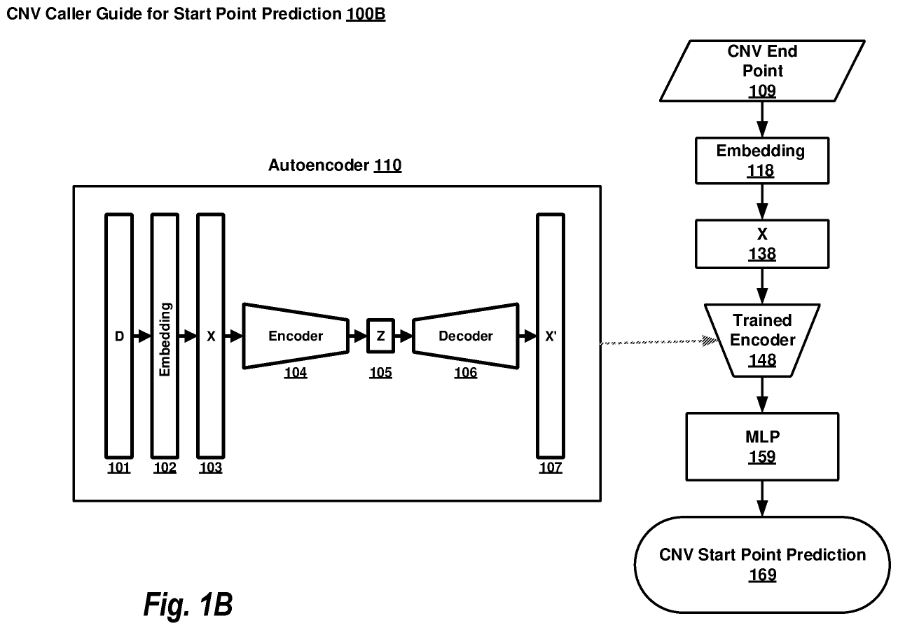

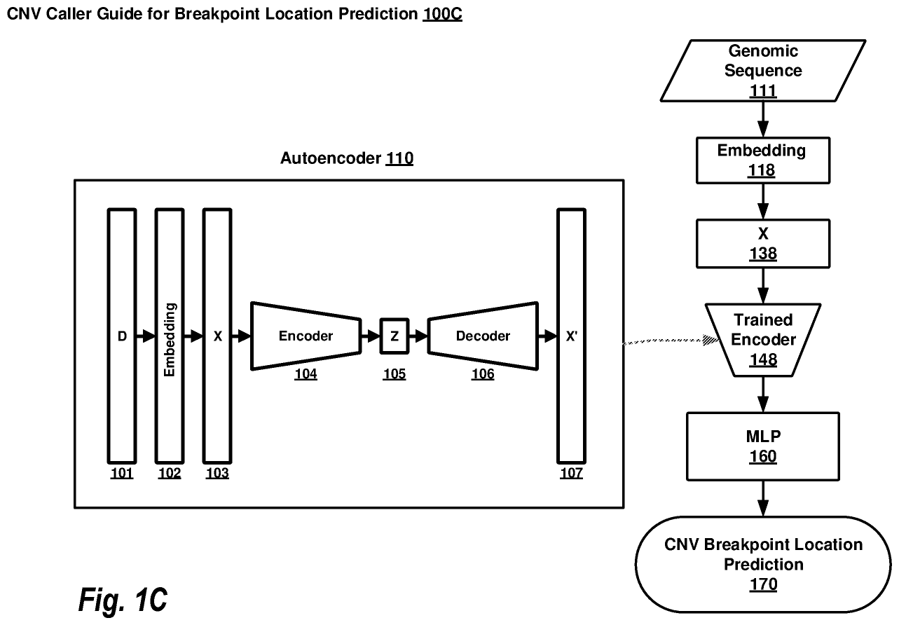

The discussion now turns to a discussion of the components of the technology disclosed. First, the training of a CNV caller guide model is discussed. Next, the discussion turns to the components constituting the CNV caller guide in further detail. Finally, the framework for predicting one or more CNV breakpoint locations is summarized. FIG. 1A is a schematic diagram illustrating a CNV caller guide 100A for detecting the end of a CNV breakpoint. In a first training procedure, an autoencoder 110 comprising encoder 104 and decoder 106 processes base sequence D 101 as training sequence data to generate X′ 107 as reconstructed sequence data to obtain a sequence reconstruction training error. The sequence reconstruction training error is obtained from a particular loss function (e.g., logistic regression/log loss, multi-class cross-entropy/softmax loss, binary cross-entropy loss, mean-squared error loss, L1 loss, L2 loss, smooth L1 loss, and Huber loss). Autoencoder 110 is trained iteratively through backpropagation to minimize the sequence reconstruction training error. One of skill in the art will recognize that a transformer architecture or other encoder-decoder can be substituted for the autoencoder and trained in an equivalent manner.

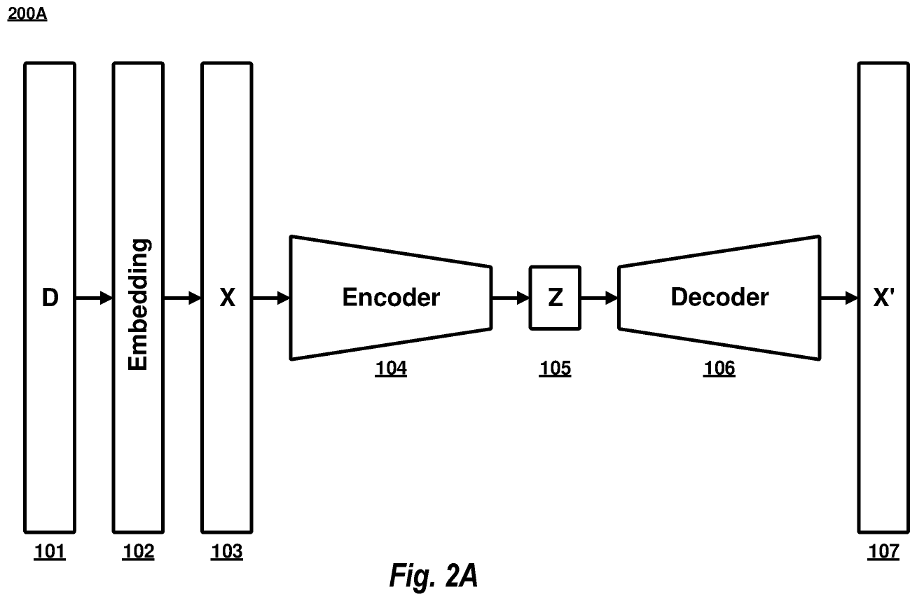

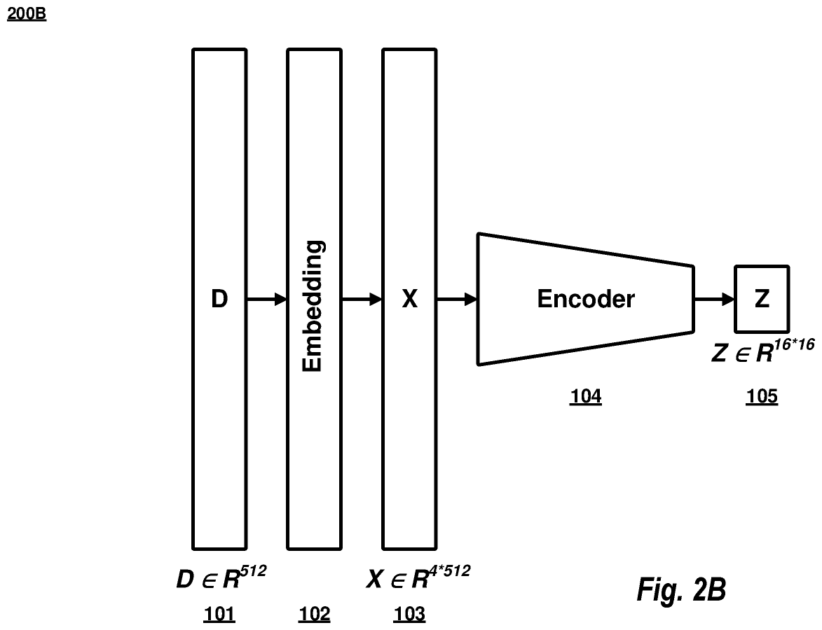

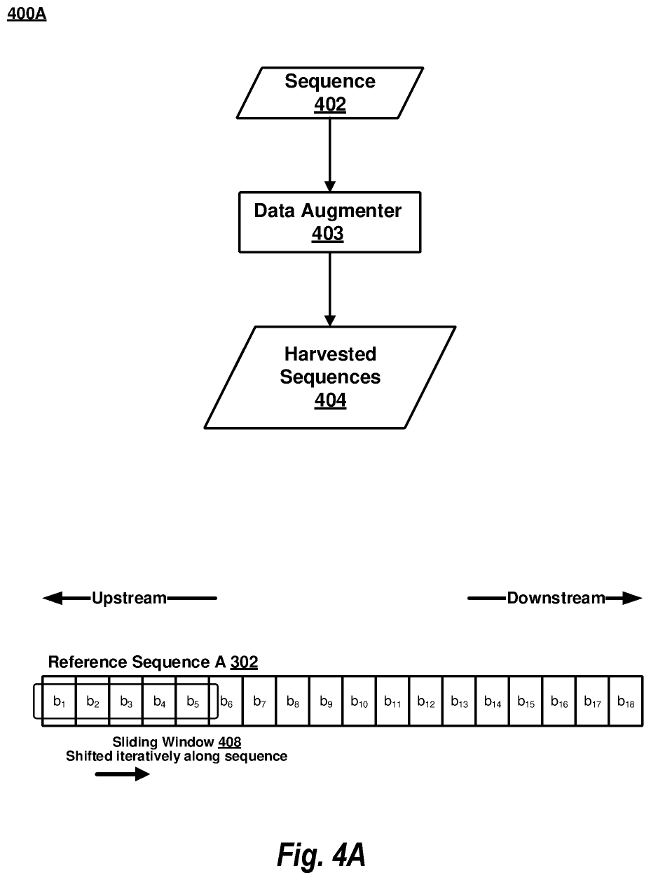

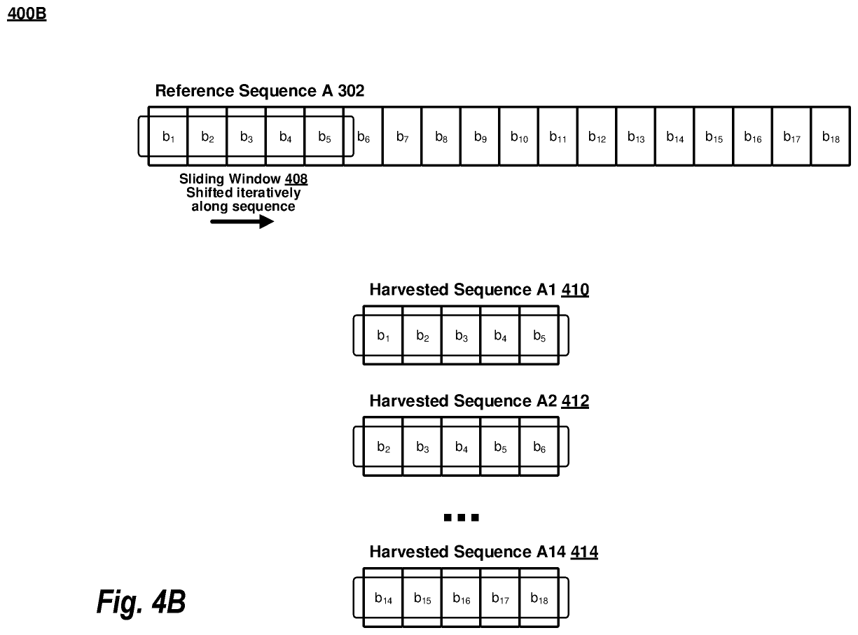

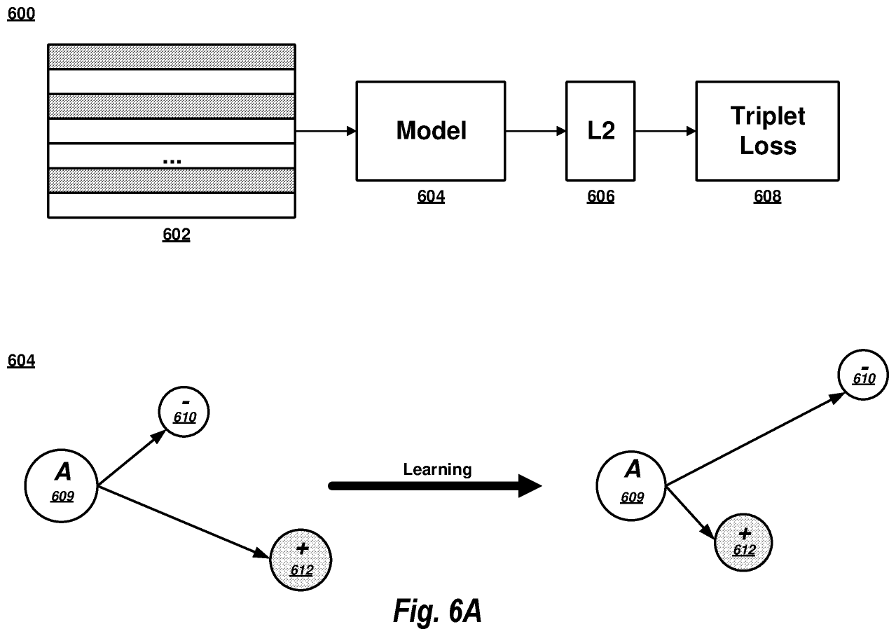

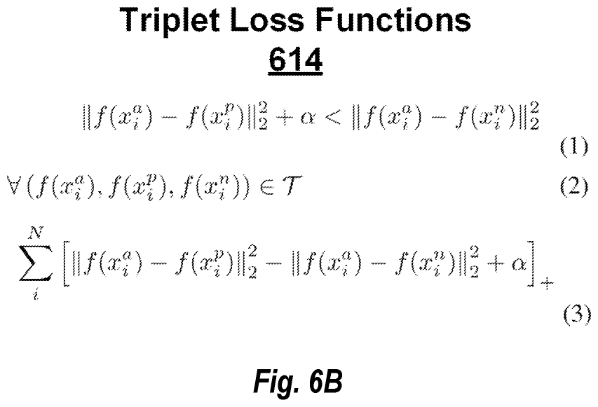

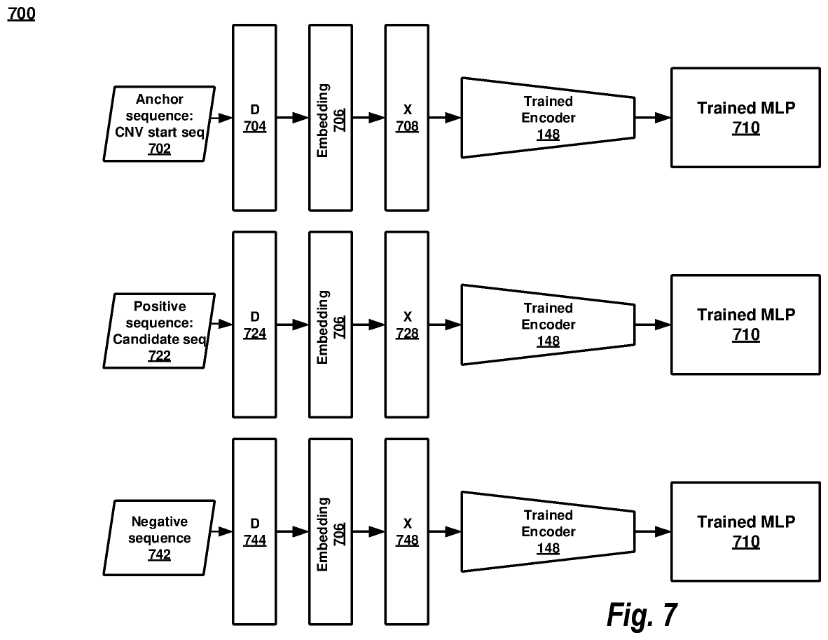

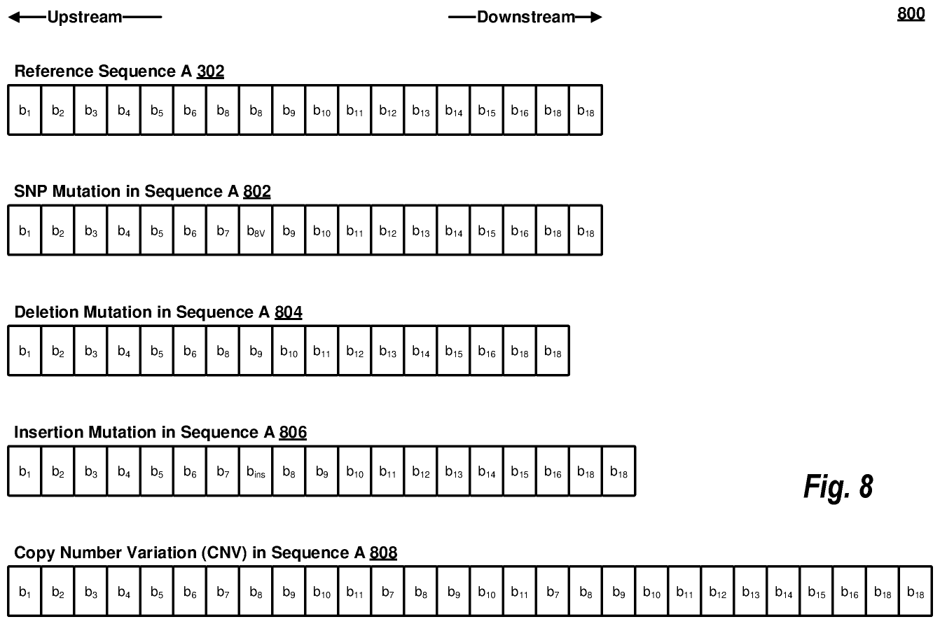

The resulting trained encoder 104 is implemented into the disclosed CNV caller guide 100A for end point prediction. Data corresponding to a CNV start point 108, wherein the location of the start of the CNV breakpoint in the sequence data is known, undergoes an embedding logic 118 to generate data X 138. Data X 138 is processed by the trained encoder 148 and a multi-layer perceptron 158 to generate learned representations of the input sequence data corresponding to CNV start point 108 and determine a similarity between the CNV start point 108 sequence and a particular subject candidate sequence to predict whether or not the particular subject candidate sequence is likely to contain the end of the CNV breakpoint. FIGS. 2A and 2B illustrate the alternative representation of input genomic sequence data in further detail, while FIGS. 7 and 8 illustrate CNV breakpoint locations in further detail. Generation of training data corresponding to the CNV start point 108 is elaborated upon further in FIGS. 4A and 4B. The training process for CNV caller guide 100A is elaborated upon further in FIGS. 6A and 6B.

The CNV caller guide 100A for end point prediction is trained on CNV start point 108 training data to learn the CNV end point prediction 168 by processing a CNV breakpoint training error. The CNV breakpoint training error is obtained from a particular loss function (e.g., logistic regression/log loss, multi-class cross-entropy/softmax loss, binary cross-entropy loss, mean-squared error loss, L1 loss, L2 loss, smooth L1 loss, and Huber loss). CNV caller guide 100A is trained iteratively through backpropagation to minimize the CNV breakpoint training error.

In another implementation of the technology disclosed, the start of a particular CNV breakpoint is predicted from the known location of the end of the particular CNV breakpoint. In comparison to FIG. 1A, FIG. 1B schematically illustrates the prediction of CNV start points rather than CNV end points.

FIG. 1B is a schematic diagram illustrating a CNV caller guide 100B for detecting the start of a CNV breakpoint. In a first training procedure, as seen in FIG. 1A, the autoencoder 110 processes based sequence D 101 as training sequence data to obtain X′ 107 as sequence reconstruction data. The sequence reconstruction training error is obtained from a particular loss function (e.g., logistic regression/log loss, multi-class cross-entropy/softmax loss, binary cross-entropy loss, mean-squared error loss, L1 loss, L2 loss, smooth L1 loss, and Huber loss). Autoencoder 110 is trained iteratively through backpropagation to minimize the sequence reconstruction training error.

The resulting trained encoder 148 is implemented into the disclosed CNV caller guide 100B. Data corresponding to a CNV end point 109, wherein the location of the start of the CNV breakpoint in the sequence data is known, undergoes an embedding logic 118 to generate data X 138. Data X 138 is processed by the trained encoder 148 and a multi-layer perceptron 159 to generate learned representations of the input sequence data corresponding to CNV end point 109 and determine a similarity between the CNV end point 109 sequence and a particular subject candidate sequence to predict whether or not the particular subject candidate sequence is likely to contain the start of the CNV breakpoint.

As seen in FIG. 1B, the CNV caller guide 100B for start point prediction is trained on CNV end point 109 training data to learn the CNV end point prediction 169 by processing a CNV breakpoint training error. In contrast to CNV caller guide 100A, the CNV breakpoint training error obtained from training the CNV caller guide 100B is the error respective to detecting CNV start points. The CNV breakpoint training error is obtained from a particular loss function (e.g., logistic regression/log loss, multi-class cross-entropy/softmax loss, binary cross-entropy loss, mean-squared error loss, L1 loss, L2 loss, smooth L1 loss, and Huber loss). CNV caller guide 100B is trained iteratively through backpropagation to minimize CNV breakpoint training error. Generation of training data corresponding to the CNV end point 109 is elaborated upon further in FIGS. 4A and 4B. The training process for CNV caller guide 100B is elaborated upon further in FIGS. 6A and 6B.

In another implementation of the technology disclosed, the start and end of a particular CNV breakpoint is predicted from a genomic sequence. In comparison to FIG. 1A and FIG. 1B, FIG. 1C schematically illustrates the prediction of both the start and end of CNV breakpoints.

FIG. 1C is a schematic diagram illustrating a CNV caller guide 100C for breakpoint location prediction for detecting the location of a CNV breakpoint. In a first training procedure, as seen in FIGS. 1A and 1B, the autoencoder 110 processes base sequence D 101 as training sequence data to obtain X′ 107 as sequence reconstruction data. The sequence reconstruction training error is obtained from a particular loss function (e.g., logistic regression/log loss, multi-class cross-entropy/softmax loss, binary cross-entropy loss, mean-squared error loss, L1 loss, L2 loss, smooth L1 loss, and Huber loss). Autoencoder 110 is trained iteratively through backpropagation to minimize the sequence reconstruction training error.

The resulting trained encoder 148 is implemented into the CNV caller guide 100B. Data corresponding to a genomic sequence 111, wherein the location of the start of the CNV breakpoint in the sequence data is known, undergoes an embedding logic 118 to generate data X 138. Data X 138 is processed by the trained encoder 148 and a multi-layer perceptron 160 to generate learned representations of the input sequence data corresponding to CNV start point 108 and determine a similarity between the CNV start point 109 sequence and a particular subject candidate sequence to predict whether or not the particular subject candidate sequence is likely to contain the location of a CNV breakpoint, comprising both start and end points.

As seen in FIG. 1C, the CNV caller guide 100C for breakpoint location prediction is trained on genomic sequence 111 training data to learn the CNV breakpoint location prediction 170 by processing a CNV breakpoint training error. In contrast to CNV caller guide 100A and CNV caller guide 100B, the CNV breakpoint training error obtained from training the CNV caller guide 100C is the error respective to detecting the CNV breakpoint location. The CNV breakpoint training error is obtained from a particular loss function (e.g., logistic regression/log loss, multi-class cross-entropy/softmax loss, binary cross-entropy loss, mean-squared error loss, L1 loss, L2 loss, smooth L1 loss, and Huber loss). CNV caller guide 100C is trained iteratively through backpropagation to minimize CNV breakpoint training error. Generation of training data corresponding to the genomic sequence 111 is elaborated upon further in FIGS. 4A and 4B. The training process for CNV caller guide 100C is elaborated upon further in FIGS. 6A and 6B.

In other implementations, alternative model may be implemented to achieve the same goal of generating an alternate representation of a genomic sequence, or a prediction of CNV breakpoints. One skilled in the art will appreciate that the encoder model and/or the CNV caller guide can use various padding and striding configurations. It can use different output functions (e.g., classification or regression) and may or may not include one or more fully-connected layers. It can use 1D convolutions, 2D convolutions, 3D convolutions, dilated or atrous convolutions, transpose convolutions, depthwise separable convolutions, pointwise convolutions, 1×1 convolutions, group convolutions, flattened convolutions, spatial and cross-channel convolutions, shuffled grouped convolutions, spatial separable convolutions, and deconvolutions. It can use one or more loss functions such as logistic regression/log loss, multi-class cross-entropy/softmax loss, binary cross-entropy loss, mean-squared error loss, L1 loss, L2 loss, smooth L1 loss, and Huber loss. In extended implementations, it could be extended to 4D or 5D convolutions. It can use any parallelism, efficiency, and compression schemes such TFRecords, compressed encoding (e.g., PNG), sharding, parallel calls for map transformation, batching, prefetching, model parallelism, data parallelism, and synchronous/asynchronous SGD. It can include upsampling layers, downsampling layers, recurrent connections, gates and gated memory units (like an LSTM or GRU), residual blocks, residual connections, highway connections, skip connections, peephole connections, activation functions (e.g., non-linear transformation functions like rectifying linear unit (ReLU), leaky ReLU, exponential liner unit (ELU), sigmoid and hyperbolic tangent (tanh)), batch normalization layers, regularization layers, dropout, pooling layers (e.g., max or average pooling), global average pooling layers, and attention mechanisms. Again, a transformer or other encoder-decoder could be substituted for an autoencoder.

### Encoder

Next, the Application describes the trained encoder in further detail. The technology disclosed comprises an encoder to address dimensionality and data size issues commonplace to genomic data analysis. FIG. 2A is a schematic diagram of an autoencoder 200A for sequence compression and sequence reconstruction. Base sequence D 101 (such as a DNA sequence) undergoes embedding process 102 to generate data X 103. Data X 103 is processed by encoder 104 to generate a learned representation Z 105 of base sequence D 101 within a latent space. Learned representation Z 105 may be decoded from the latent space by decoder 106 to generate X′ 107, which is a reconstructed version of base sequence D 101. Error may be measured for autoencoder 200A by determining similarity of X 103 and X′ 107.

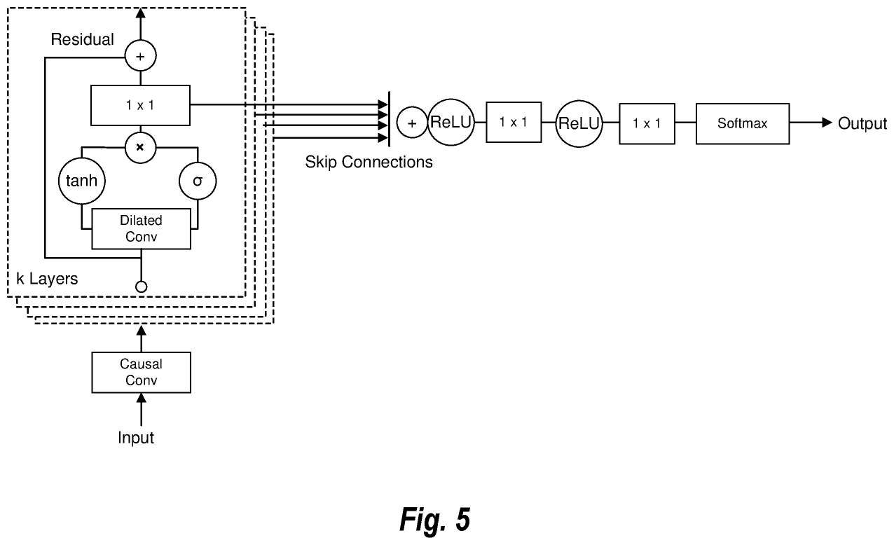

In some implementations of the technology disclosed, the autoencoder comprises a modified WaveNet structure. Briefly, the autoencoder comprises groups of residual blocks, and skip connections, wherein each group of residual blocks is parameterized by a number of convolution filters in the residual blocks, a convolution window size of the residual blocks, and an atrous (dilated) convolution rate of the residual blocks. The convolution window size and atrous convolution rate vary between the groups of residual blocks. Base sequence D 101 (as training sequence data) includes pairs of translated sequences generated from benign variants and pathogenic variants and used as benign training examples and as pathogenic training examples. Model architecture is described further in FIG. 5.

The trained encoder 148 is pre-trained as part of an autoencoder that is trained to encode a base sequence into a learned representation of the base sequence. In most implementations of the technology disclosed, the autoencoder is trained using unsupervised learning. The resulting encoder from autoencoder 200A is shown in FIG. 2B.

FIG. 2B is a schematic diagram 200B of an encoder for sequence compression. This Application defines sequence compression as the encoding of DNA sequence information into lower-dimensionality space. Base sequence D 101 (such as a DNA sequence) undergoes embedding process 102 to generate data X 103. Whereas base sequence D 101 has dimensionality of 512, data X 103 has dimensionality 4*512 to account for four possible nucleic acid bases. Data X 103 is processed by encoder 104 to generate a learned representation Z 105 of base sequence D 101 within a latent space. Learned representation Z 105 possesses a lower dimensionality of 16*16. The Application will now cover embedding of genomic sequence data using one-hot encoding.

### Sequence Embedding

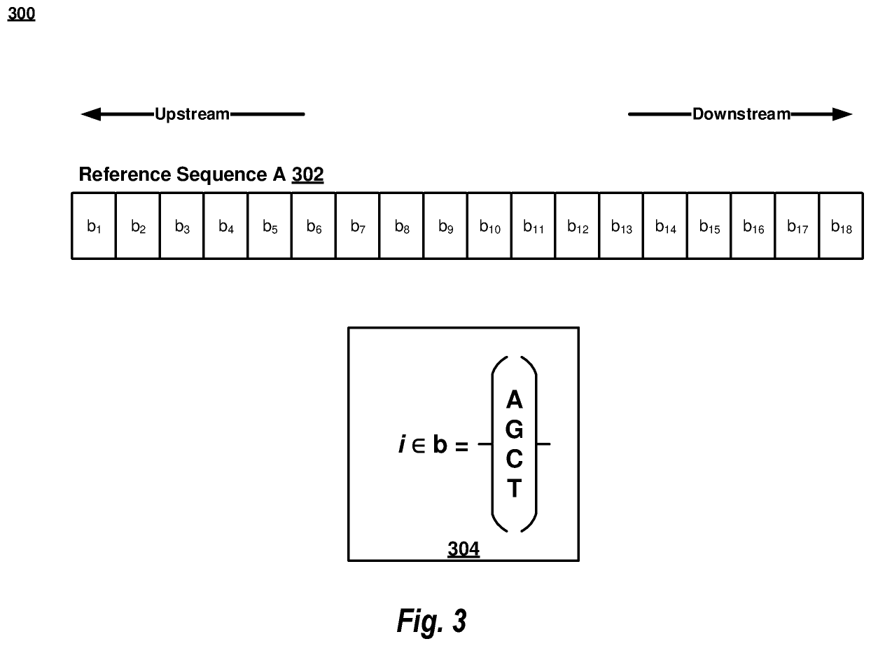

FIG. 3 illustrates a schematic diagram 300 of the vectorization of a reference genomic sequence. Reference Sequence A 302 comprises eighteen nucleic acid bases b. A person skilled in the art will recognize that genomic sequences are typically substantially larger than eighteen bases and Reference Sequence A 302 is shown purely as an educational example with a short length for clarity. The closer to the initial start point of Reference Sequence A 302 a nucleic acid base is, the further upstream the nucleic acid base is. Contrarily, the further from the initial start point of Reference Sequence A 302 a nucleic acid base is, the further downstream the nucleic acid base is. For example, nucleic acid base b6 is downstream respective to nucleic acid b2, but nucleic acid base b6 is upstream respective to nucleic acid b15. A particular nucleic acid base b may be an adenine (A), guanine (G), cytosine (C), or thymine (T) nucleic acid. In some implementations of the technology disclosed, embedding process 102 may include an additional dimension for an unknown or atypical nucleic acid base N. Equation 304 illustrates a vector corresponding to each nucleic acid base within Reference Sequence A 302, wherein each nucleic acid base will comprise a ‘1’ in the vector entry corresponding to the correct nucleic acid and a ‘0’ in all other vector entries (e.g., a cytosine nucleic acid will be embedded via one-hot encoded as [0, 0, 1, 0] using equation 304).

### Sequence Augmentation

The above described autoencoder 200A is trained using a sufficiently large dataset comprising genomic data. However, data corresponding specifically to CNV breakpoints is more scarce and requires a data augmentation process to generate sufficient data for training. To generate enough training data for CNV caller guide 100A, CNV caller guide 100B, and CNV caller guide 100C, augmented sequences are harvested by sliding over the real pairs of sequences at upstream and downstream offsets from the observed CNV breakpoint starts and ends.

FIG. 4A is a schematic diagram 400A illustrating a system for harvesting augmented sequences from a real reference sequence. Data augmenter 403 processes a particular genomic sequence 402 to generate harvested sequences 404. For example, consider Reference Sequence A 302. A sliding window 408 comprising five nucleic acid bases can be shifted iteratively along the sequence. This process is shown in more detail in FIG. 4B. A person skilled in the art will appreciate that the small size of Reference Sequence A 302 and sliding window 408 are shown for simplicity, whereas the sequence length, sliding window size, and window step size will be much larger in real world scenarios.

FIG. 4B illustrates a schematic diagram 400B of a plurality of harvested sequences from a real reference sequence. As sliding window 408 slides over Reference Sequence A 302, smaller sub-sequences are harvested. In the shown example, sliding window 408 has a window size of five and a step size of one. Thus, the first harvested sequence A1 410 comprises nucleic acid bases b1:b5. Harvested sequence A2 412 comprises nucleic acid bases b2:b6. This process is repeated iteratively, where sliding window 408 slides one base at a time, until sliding window 408 reaches harvested sequence A14 414, comprising nucleic acid bases b14:b18. The sliding window 408 cannot proceed any further, as there will no longer be five base pairs to fill the window.

### Triplet Loss Model

Generation of harvested sequences for regions upstream and downstream of CNV breakpoints allows for generalizable training of a CNV caller guide. The CNV caller guide is trained by processing three sequences corresponding to a CNV breakpoint: an anchor sequence, a positive sequence, and a negative sequence. In one implementation of the technology disclosed, the anchor sequence comprises the start of the CNV breakpoint, the positive sequence comprises the end of the CNV breakpoint, and the negative sequence comprises an unrelated downstream section of the genomic sequence. The CNV caller guide is trained using a triplet loss function to minimize the distance between the anchor sequence and the positive sequence, and maximize the distance between the anchor sequence and the negative sequence.

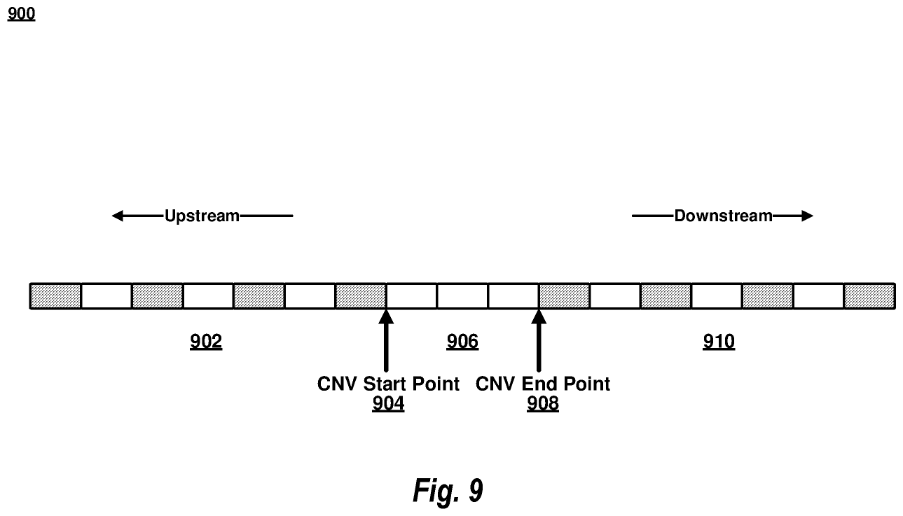

The trained CNV caller guide determines a similarity (homology) between an anchor sequence and a subject candidate sequence, where the subject candidate sequence is a sequence downstream to the anchor sequence, by comparing the learned representation of the anchor sequence against the learned representation of the subject candidate sequence generated by the trained encoder 148. The triplet loss model and CNV caller guide are described in further detail in FIGS. 6A, 6B, and 9.

In some implementations of the technology disclosed, the CNV caller guide comprises an encoder with an augmented WaveNet structure. The WaveNet is a deep neural network for generating raw audio waveforms. The WaveNet distinguishes itself from other convolutional networks since it is able to take relatively large ‘visual fields’ at low cost. Moreover, it is able to add conditioning of the signals locally and globally, which allows the WaveNet to be used as a text to speech (TTS) engine with multiple voices, is the TTS gives local conditioning and the particular voice the global conditioning.

The main building blocks of the WaveNet are the causal dilated convolutions. As an extension on the causal dilated convolutions, the WaveNet also allows stacks of these convolutions, as shown in FIG. 5. To obtain the same receptive field with dilated convolutions in this figure, another dilation layer is required. The stacks are a repetition of the dilated convolutions, connecting the outputs of dilated convolution layer to a single output. This enables the WaveNet to get a large ‘visual’ field of one output node at a relatively low computational cost. For comparison, to get a visual field of 512 inputs, a fully convolutional network (FCN) would require 511 layers. In the case of a dilated convolutional network, we would need eight layers. The stacked dilated convolutions only need seven layers with two stacks or six layers with four stacks. To get an idea of the differences in computational power required for covering the same visual field, the following table shows the number of weights required in the network with the assumption of one filter per layer and a filter width of two. Furthermore, it is assumed that the network is using binary encoding of the 8 bits.

| Network | No. | No. of weights | Total No. of |
|---|---|---|---|
| type | stacks | per channel | weights |
| FCN | 1 | 2.6.105 | 2.6.106 |
| WN | 1 | 1022 | 8176 |
| WN | 2 | 1022 | 8176 |
| WN | 4 | 508 | 4064 |

The WaveNet adds a skip connection before the residual connection is made, which bypasses following residual blocks. Each of these skip connections is summed before passing them through a series of activation functions and convolutions. Intuitively, this is the sum of the information extracted in each layer.

The CNV caller guide measures similarity between the anchor sequence and the subject candidate sequence using a distance score. In some implementations of the technology disclosed, the distance score is based on L2 norm Euclidean distance. In other implementations of the technology disclosed, the distance score may be based on an alternative distance measure such as Manhattan distance or Hamming distance. The CNV caller guide is trained on a training dataset that comprises pairs of anchor and positive sequences that contain CNV breakpoint starts and ends, and negative sequences that do not contain the CNV breakpoint ends. The CNV caller guide is trained to determine similarity between paired anchor and positive sequences and dissimilarity between the anchor sequences and the negative sequences by comparing the learned representations of the anchor sequences against the learned representations of the positive sequences and the negative sequences, using a triplet loss function.

FIG. 6A is a schematic diagram of a triplet loss model. In system 600, data 602 is processed by a model 604. In some implementations of the technology disclosed, model 604 is a multi-layer perceptron. However, in other implementations of the technology disclosed, the model may comprise a wide range of additional machine learning and deep learning architectures (e.g., logistic regression, linear regression, random forest, convolutional neural networks, recurrent neural networks, and so on). Following processing by model 604, distances generated by the model 604 are measured based on an L2 norm Euclidean distance 606 to produce a triplet loss 608. The triplet loss 608 determined by a triplet loss function is zero when a distance between a pair of anchor and positive sequences plus a margin is less than a distance between the anchor sequence and a negative sequence. The triplet loss 608 is non-zero when the distance between the pair of anchor and positive sequences plus the margin is more than the distance between the anchor sequence and the negative sequence. The margin may take on a plurality of values. In some implementations of the technology disclosed, the margin is equivalent to two.

FIG. 6B shows an equation for triplet loss functions 614. The embedding is represented by f(x)∈Rd. It embeds an input x into a d-dimensional Euclidean space. Additionally, the embedding is constrained to live on the d-dimensional hypersphere, i.e., ∥f(x)∥2=1. This loss is motivated in the context of nearest-neighbor classification. Here, the model 604 aims to ensure that anchor input x a i (anchor) of a specific sequence is closer to all other positive sequences x p i (positive) than it is to any negative sequence x n i. As shown in FIG. 6A, for example, the model 604 learns to ensure that the anchor input 609 (e.g., anchor sequence) is closer in terms of distance to positive sequences 612 than the anchor input 609 is to negative sequences 610 by determining the triplet loss 608.

Additionally, α is a margin that is enforced between positive and negative pairs. T is the set of all possible triplets in the training set and has cardinality N. The loss that is being minimized is shown in the third equation of the triplet loss functions 614.

### Copy Number Variation Caller Guide

FIG. 7 is a schematic diagram of a so-called Siamese CNV caller guide. That is, the structures and coefficients of the three processing arms are identical for the task undertaken. System 700 comprises each component from the previously discussed figures within the overall framework of the CNV caller guide model. In the displayed implementation, the anchor sequence 702 is a CNV start point, but in other implementations, this may also be a CNV end point. Coefficients of the multi-layer perceptron 710 will depend on which task (start point, end point or breakpoint) is being processed. The positive sequence 722 is a subject candidate sequence. The negative sequence 742 is a downstream, highly dissimilar sequence to the anchor sequence 702. To determine the subject candidate sequence most likely to contain the CNV end point, the model is iteratively run along the genomic sequence downstream to test all possible subject candidate sequences until one is determined by the trained CNV caller guide to be highly likely (as determined by a specific distance threshold) to contain the CNV end point.

Each input sequence—that is, the anchor sequence 702, the positive sequence 722, and the negative sequence 742—is processed as input 704, 724, and 744, respectively. Inputs are embedded by process 706, generating one-hot encoded vectors 708, 728, and 748, respectively. Each embedded input is simultaneously processed by the same trained encoder 148 and trained multi-layer perceptron (MLP) (or, in other implementations, alternative machine learning model). The output is one when the end of the CNV breakpoint is located on the subject candidate sequence (i.e., the distance score is below a distance threshold), and the output is zero when the end of the CNV breakpoint is not located on the subject candidate sequence (i.e., the distance score is above a distance threshold).

In one implementation of the technology disclosed, the distance threshold is determined based on the trained CNV caller guide's performance on a validation set. In some implementations, the distance threshold is based on an average of a mean of distances determined by the trained CNV caller guide between pairs of anchor and positive sequences in the validation set and a mean of distances determined by the trained CNV caller guide between the anchor sequences and negative sequences in the validation set. In other implementations, the distance threshold is based on an average of upper n percentiles of the distances determined by the trained CNV caller guide between the pairs of anchor and positive sequences in the validation set and lower n percentiles of the distances determined by the trained CNV caller guide between the anchor sequences and negative sequences in the validation set.

### Copy Number Variation Structure

The discussion thus far has covered the components of the technology disclosed, as well as the methods by which the models are trained. While the disclosed system is described herein with reference to particular blocks, it is to be understood that the blocks are defined for convenience of description and are not intended to require a particular physical arrangement of component parts. Further, the blocks need not correspond to physically distinct components. To the extent that physical distinct components are used, connections between components can be wired and/or wireless as desired. The different elements or components can be combined into single software modules and multiple software modules can run on the same hardware.

Now, the discussion turns to explain the input and output data in further detail. FIG. 8 illustrates a schematic diagram 800 of examples of genomic variation. Reference Sequence A 302 is again shown with eighteen base pairs, marked by the upstream direction and the downstream direction. Mutated sequence 802 illustrates a single nucleotide polymorphism (SNP) mutation in Sequence A, wherein nucleic acid base b8 has mutated to nucleic acid base b8v. Mutated sequence 804 illustrates a deletion mutation in Sequence A, wherein nucleic acid base b7 has been deleted from the sequence entirely. Mutated sequence 806 illustrates an insertion mutation in Sequence A, wherein nucleic acid base bins has been inserted in between b7 and b8.

In contrast to mutated sequences 802, 804, and 806, sequence 808 comprises a small repeating CNV. A person skilled in the art will recognize that the length of CNVs detected by the technology disclosed is much larger at one kB or higher, whereas sequence 808 is purely given as an educational example. Sequence 808 is longer than the reference sequence A 302 because it comprises a repeating section of nucleic acids b7:b11.

While FIG. 8 displays various types of mutations in small sequence segments, FIG. 9 illustrates an example CNV breakpoint in context of a longer genomic segment. Each block shown may represent any number of nucleic acids of any length. All blocks 902 are upstream of the CNV breakpoint 906 and all blocks 910 are downstream of the CNV breakpoint 906. The CNV breakpoint 906 is marked by a CNV start point 904 and a CNV end point 908. In some implementations of the technology disclosed, a trained CNV caller guide processes the CNV start point 904 to generate a prediction of the location of CNV end point 908. In other implementations of the technology disclosed, a trained CNV caller guide processes the CNV end point 908 to generate a prediction of the location of CNV start point 904. In yet another implementation of the technology disclosed, a trained CNV caller guide processes sequence 900 to generate a prediction of the locations of both CNV start point 904 and CNV end point 908, therefore predicting the overall location and size of the CNV breakpoint 906.

### Performance Measure Results as Objective Indicia of Non-Obviousness and Inventiveness

Thus far, the description has encompassed a method of processing sequence data comprising a known location of the start of a copy number variant breakpoint to generate a prediction for the location of the end of the copy number variant breakpoint. The discussion now turns to performance results of various implementations of the technology disclosed.

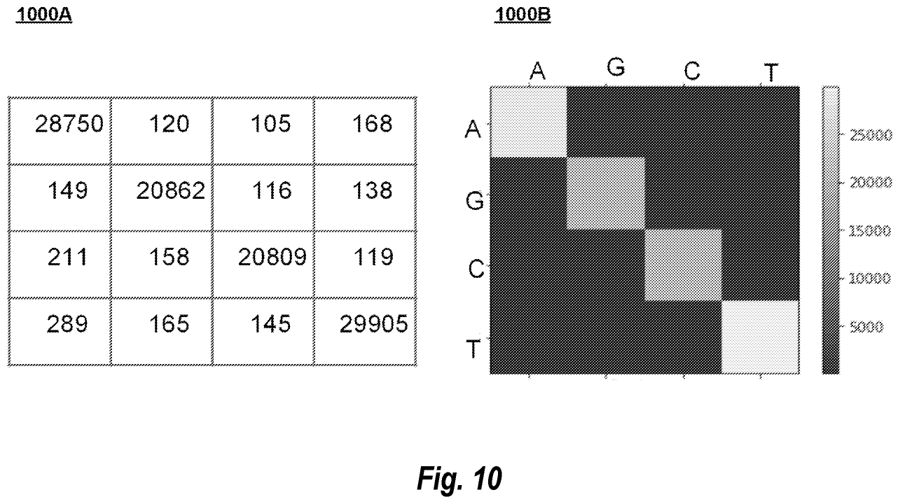

FIG. 10 contains performance results from the disclosed autoencoder, in accordance with one implementation of the technology disclosed. Graph 1000A comprises a confusion matrix wherein the diagonal elements show the number of correct sequence nucleic acid base reconstruction and off-diagonal elements show the number of incorrect sequence nucleic acid base reconstruction. Graph 1000B comprises the same confusion matrix as a heat map, wherein the spectrum from purple to yellow is proportional to element count such that yellow is maximum and purple is minimum. The autoencoder overall has an accuracy equal to 0.983.

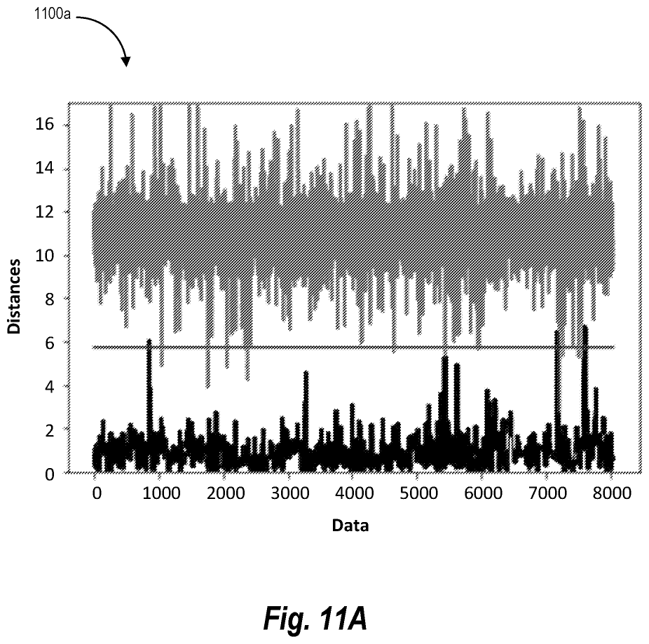

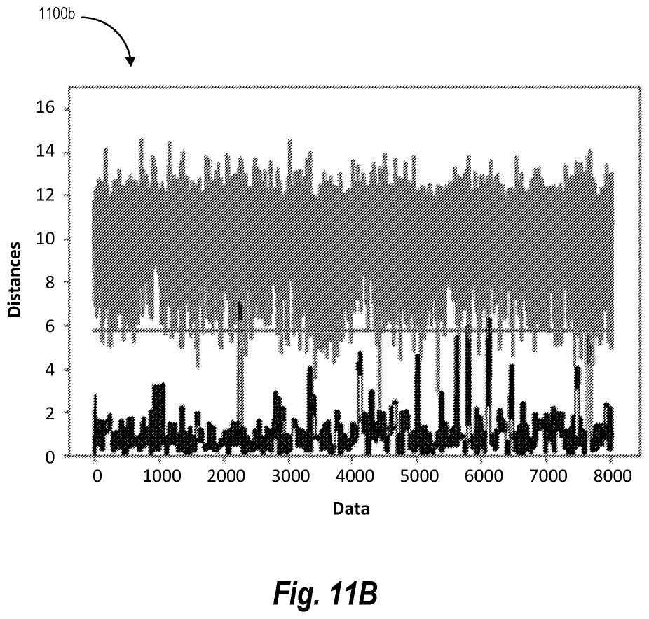

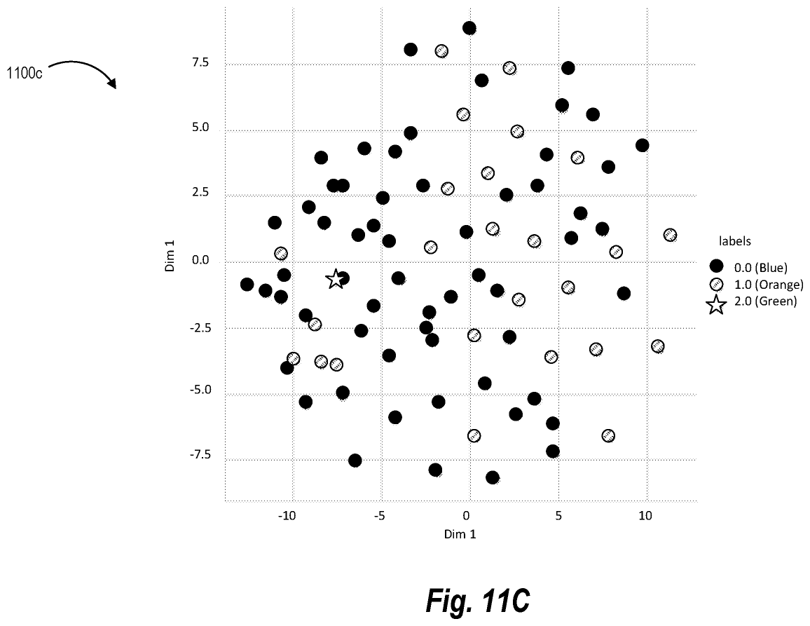

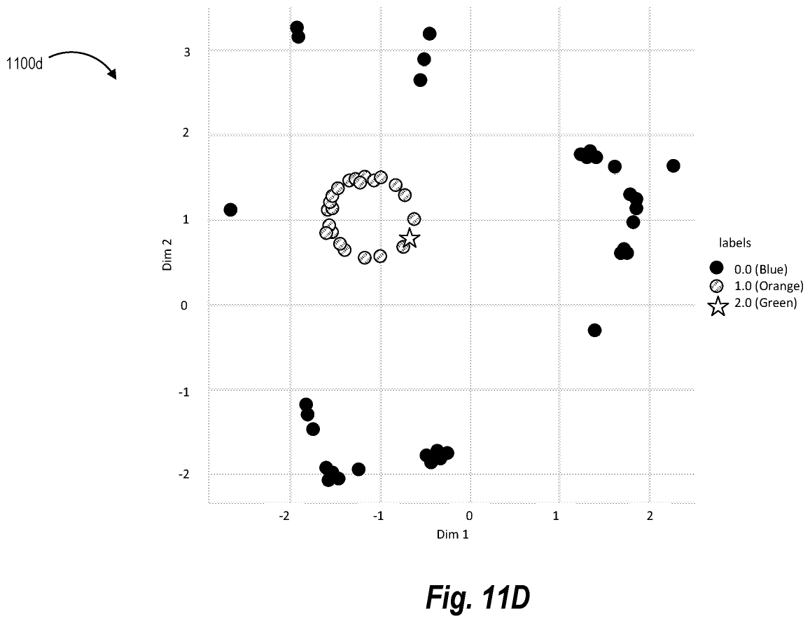

FIGS. 11A-11D contain performance results from the disclosed triplet models. Following training of the triplet model, the test set is split into a validation set and a test subset. The trained model performance on the validation set is shown in graph 1100A of FIG. 11A to determine the threshold line (i.e., black line) while grey and black curves show the distance between positive and anchor and the distance between negative and anchor, respectively. Using the threshold, the trained triplet model was run on the test subset (shown in graph 1100B of FIG. 11B) to label them as positive or negative using the threshold line (i.e., black line). During training, the triplet loss function is trying to minimize the distance between positive data and anchor while maximizing the distance between anchor and negative data. The loss function is to minimize max(0, distance(anchor, positive)+margin−distance(anchor, negative). At test time, we have the margin between black curve and grey curve shown in graph 1100B. Additionally, t-SNE dimension reduction demonstrates how the trained triplet model is trying to project the anchor (green dot), its corresponding augmented positive data (orange dots) close in proximity to each other while pushing the negative data (blue dots) further away. In FIG. 11C, graph 1100C shows the spread of the randomly selected CNVs within the test data without processing via the disclosed triplet model. In FIG. 11D, graph 1100D shows the performance of triplet model on the test data.

### Computer System

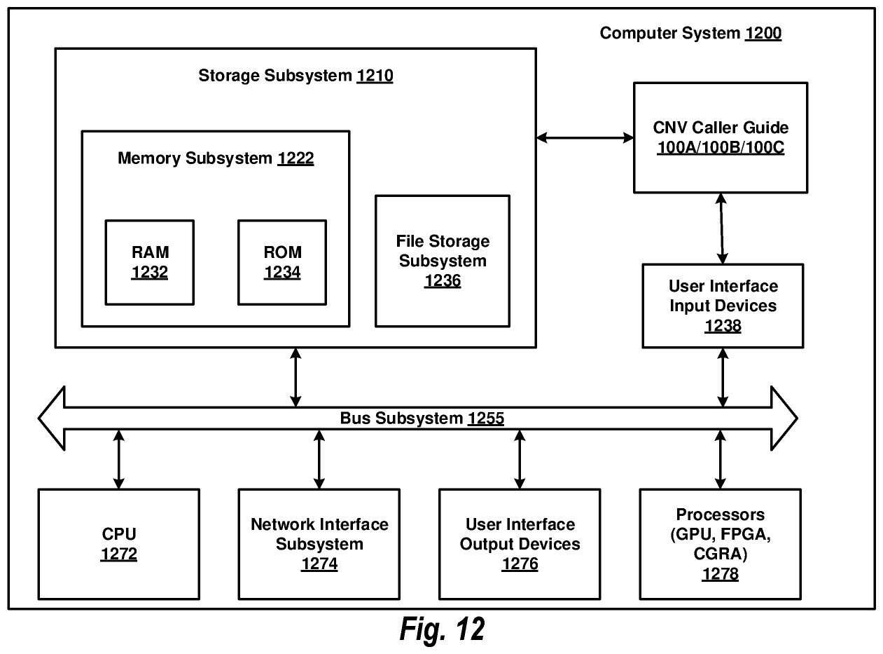

FIG. 12 is a computer system 1200 that can be used to implement the technology disclosed. Computer system 1200 includes at least one central processing unit (CPU) 1272 that communicates with a number of peripheral devices via bus subsystem 1255. These peripheral devices can include a storage subsystem 1210 including, for example, memory devices and a file storage subsystem 1236, user interface input devices 1238, user interface output devices 1276, and a network interface subsystem 1274. The input and output devices allow user interaction with computer system 1200. Network interface subsystem 1274 provides an interface to outside networks, including an interface to corresponding interface devices in other computer systems.

In one implementation, the CNV Caller Guide 100A/100B/100C is communicably linked to the storage subsystem 1210 and the user interface input devices 1238.

User interface input devices 1238 can include a keyboard; pointing devices such as a mouse, trackball, touchpad, or graphics tablet; a scanner; a touch screen incorporated into the display; audio input devices such as voice recognition systems and microphones; and other types of input devices. In general, use of the term “input device” is intended to include all possible types of devices and ways to input information into computer system 1200.

User interface output devices 1276 can include a display subsystem, a printer, a fax machine, or non-visual displays such as audio output devices. The display subsystem can include an LED display, a cathode ray tube (CRT), a flat-panel device such as a liquid crystal display (LCD), a projection device, or some other mechanism for creating a visible image. The display subsystem can also provide a non-visual display such as audio output devices. In general, use of the term “output device” is intended to include all possible types of devices and ways to output information from computer system 1200 to the user or to another machine or computer system.

Storage subsystem 1210 stores programming and data constructs that provide the functionality of some or all of the modules and methods described herein. These software modules are generally executed by processors 1278.

Processors 1278 can be graphics processing units (GPUs), field-programmable gate arrays (FPGAs), application-specific integrated circuits (ASICs), and/or coarse-grained reconfigurable architectures (CGRAs). Processors 1278 can be hosted by a deep learning cloud platform such as Google Cloud Platform™, Xilinx™, and Cirrascale™. Examples of processors 1278 include Google's Tensor Processing Unit (TPU)™, rackmount solutions like GX4 Rackmount Series™, GX15 Rackmount Series™, NVIDIA DGX-1™, Microsoft' Stratix V FPGA™, Graphcore's Intelligent Processor Unit (IPU)™, Qualcomm's Zeroth Platform™ with Snapdragon Processors™, NVIDIA's Volta™, NVIDIA's DRIVE PX™, NVIDIA's JETSON TX1/TX2 MODULE™, Intel's Nirvana™, Movidius VPU™, Fujitsu DPI™, ARM's DynamicIQ™, IBM TrueNorth™, Lambda GPU Server with Testa V100s™, and others.

Memory subsystem 1222 used in the storage subsystem 1210 can include a number of memories including a main random-access memory (RAM) 1232 for storage of instructions and data during program execution and a read only memory (ROM) 1234 in which fixed instructions are stored. A file storage subsystem 1236 can provide persistent storage for program and data files, and can include a hard disk drive, a floppy disk drive along with associated removable media, a CD-ROM drive, an optical drive, or removable media cartridges. The modules implementing the functionality of some implementations can be stored by file storage subsystem 1236 in the storage subsystem 1210, or in other machines accessible by the processor.

Bus subsystem 1255 provides a mechanism for letting the various components and subsystems of computer system 1200 communicate with each other as intended. Although bus subsystem 1255 is shown schematically as a single bus, alternative implementations of the bus subsystem can use multiple busses.

Computer system 1200 itself can be of varying types including a personal computer, a portable computer, a workstation, a computer terminal, a network computer, a television, a mainframe, a server farm, a widely-distributed set of loosely networked computers, or any other data processing system or user device. Due to the ever-changing nature of computers and networks, the description of computer system 1200 depicted in FIG. 12 is intended only as a specific example for purposes of illustrating the preferred implementations of the present invention. Many other configurations of computer system 1200 are possible having more or less components than the computer system depicted in FIG. 12.

Each of the processors or modules discussed herein may include an algorithm (e.g., instructions stored on a tangible and/or non-transitory computer readable storage medium) or sub-algorithms to perform particular processes. The CNV Caller Guide 100A/100B/100C is illustrated conceptually as a module, but may be implemented utilizing any combination of dedicated hardware boards, DSPs, processors, etc. Alternatively, the CNV Caller Guide 100A/100B/100C may be implemented utilizing an off-the-shelf PC with a single processor or multiple processors, with the functional operations distributed between the processors. As a further option, the modules described below may be implemented utilizing a hybrid configuration in which some modular functions are performed utilizing dedicated hardware, while the remaining modular functions are performed utilizing an off-the-shelf PC and the like. The modules also may be implemented as software modules within a processing unit.

Various processes and steps of the methods set forth herein can be carried out using a computer. The computer can include a processor that is part of a detection device, networked with a detection device used to obtain the data that is processed by the computer or separate from the detection device. In some implementations, information (e.g., image data) may be transmitted between components of a system disclosed herein directly or via a computer network. A local area network (LAN) or wide area network (WAN) may be a corporate computing network, including access to the Internet, to which computers and computing devices comprising the system are connected. In one implementation, the LAN conforms to the transmission control protocol/internet protocol (TCP/IP) industry standard. In some instances, the information (e.g., image data) is input to a system disclosed herein via an input device (e.g., disk drive, compact disk player, USB port etc.). In some instances, the information is received by loading the information, e.g., from a storage device such as a disk or flash drive.

A processor that is used to run an algorithm or other process set forth herein may comprise a microprocessor. The microprocessor may be any conventional general purpose single- or multi-chip microprocessor such as a Pentium™ processor made by Intel Corporation. A particularly useful computer can utilize an Intel Ivybridge dual-12 core processor, LSI raid controller, having 128 GB of RAM, and 2 TB solid state disk drive. In addition, the processor may comprise any conventional special purpose processor such as a digital signal processor or a graphics processor. The processor typically has conventional address lines, conventional data lines, and one or more conventional control lines.

The implementations disclosed herein may be implemented as a method, apparatus, system or article of manufacture using standard programming or engineering techniques to produce software, firmware, hardware, or any combination thereof. The term “article of manufacture” as used herein refers to code or logic implemented in hardware or computer readable media such as optical storage devices, and volatile or non-volatile memory devices. Such hardware may include, but is not limited to, field programmable gate arrays (FPGAs), application-specific integrated circuits (ASICs), complex programmable logic devices (CPLDs), programmable logic arrays (PLAs), microprocessors, or other similar processing devices. One or more implementations of the technology disclosed, or elements thereof can be implemented in the form of a computer product including a non-transitory computer readable storage medium with computer usable program code for performing the method steps indicated. Furthermore, one or more implementations of the technology disclosed, or elements thereof can be implemented in the form of an apparatus including a memory and at least one processor that is coupled to the memory and operative to perform exemplary method steps. Yet further, in another aspect, one or more implementations of the technology disclosed or elements thereof can be implemented in the form of means for carrying out one or more of the method steps described herein; the means can include (i) hardware module(s), (ii) software module(s) executing on one or more hardware processors, or (iii) a combination of hardware and software modules; any of (i)-(iii) implement the specific techniques set forth herein, and the software modules are stored in a computer readable storage medium (or multiple such media).

### Clauses

The technology disclosed, in particularly, the clauses disclosed in this section, can be practiced as a system, method, or article of manufacture. One or more features of an implementation can be combined with the base implementation. Implementations that are not mutually exclusive are taught to be combinable. One or more features of an implementation can be combined with other implementations. This disclosure periodically reminds the user of these options. Omission from some implementations of recitations that repeat these options should not be taken as limiting the combinations taught in the preceding sections—these recitations are hereby incorporated forward by reference into each of the following implementations.

One or more implementations and clauses of the technology disclosed or elements thereof can be implemented in the form of a computer product, including a non-transitory computer readable storage medium with computer usable program code for performing the method steps indicated. Furthermore, one or more implementations and clauses of the technology disclosed or elements thereof can be implemented in the form of an apparatus including a memory and at least one processor that is coupled to the memory and operative to perform exemplary method steps. Yet further, in another aspect, one or more implementations and clauses of the technology disclosed or elements thereof can be implemented in the form of means for carrying out one or more of the method steps described herein; the means can include (i) hardware module(s), (ii) software module(s) executing on one or more hardware processors, or (iii) a combination of hardware and software modules; any of (i)-(iii) implement the specific techniques set forth herein, and the software modules are stored in a computer readable storage medium (or multiple such media).

The clauses described in this section can be combined as features. In the interest of conciseness, the combinations of features are not individually enumerated and are not repeated with each base set of features. The dependent clauses, formulated to satisfy US requirements, should be considered for disclosure purposes, to teach nested multiple dependencies, as permitted by EPO, JPO and other patent offices. The reader will understand how features identified in the clauses described in this section can readily be combined with sets of base features identified as implementations in other sections of this application. These clauses are not meant to be mutually exclusive, exhaustive, or restrictive; and the technology disclosed is not limited to these clauses but rather encompasses all possible combinations, modifications, and variations within the scope of the claimed technology and its equivalents.

Other implementations of the clauses described in this section can include a non-transitory computer readable storage medium storing instructions executable by a processor to perform any of the clauses described in this section. Yet another implementation of the clauses described in this section can include a system including memory and one or more processors operable to execute instructions, stored in the memory, to perform any of the clauses described in this section.

We disclose the following clauses:

### Clause Set 1—Predicting CNV End Breakpoint

1. A computer-implemented method, including:
processing sequence data that comprises:
   a start of a copy number variation (CNV) breakpoint,
   an end of the CNV breakpoint,
      wherein a location of the start of the CNV breakpoint in the sequence data is known, and
      wherein a location of the end of the CNV breakpoint in the sequence data is unknown; and
based on the processing, determining the location of the end of the CNV breakpoint in the sequence data.

2. The computer-implemented method of clause 1, wherein the sequence data has an anchor sequence.

3. The computer-implemented method of clause 2, wherein the sequence data has a plurality of candidate sequences.

4. The computer-implemented method of clause 3, wherein candidate sequences in the plurality of candidate sequences are downstream to the anchor sequence.

5. The computer-implemented method of clause 2, wherein the start of the CNV breakpoint is located on the anchor sequence.

6. The computer-implemented method of clause 3, wherein the end of the CNV breakpoint is located on one or more of the candidate sequences.

7. The computer-implemented method of clause 4, wherein a trained CNV Caller Guide processes the anchor sequence and a subject candidate sequence as inputs and generates an output that specifies whether the end of the CNV breakpoint is located on the subject candidate sequence.

8. The computer-implemented method of clause 7, wherein the output is one when the end of the CNV breakpoint is located on the subject candidate sequence.

9. The computer-implemented method of clause 8, wherein the output is zero when the end of the CNV breakpoint is not located on the subject candidate sequence.

10. The computer-implemented method of clause 7, wherein the trained CNV Caller Guide comprises a trained encoder and a trained multi-layer perceptron.

11. The computer-implemented method of clause 10, wherein the trained CNV Caller Guide processes the anchor sequence through the trained encoder and the trained multi-layer perceptron and generates a learned representation of the anchor sequence.

12. The computer-implemented method of clause 11, wherein the trained CNV Caller Guide processes the subject candidate sequence through the trained encoder and the trained multi-layer perceptron and generates a learned representation of the subject candidate sequence.

13. The computer-implemented method of clause 12, wherein the trained CNV Caller Guide determines a similarity (homology) between the anchor sequence and the subject candidate sequence by comparing the learned representation of the anchor sequence against the learned representation of the subject candidate sequence.

14. The computer-implemented method of clause 13, wherein the trained CNV Caller Guide measures the similarity using a distance score.

15. The computer-implemented method of clause 14, wherein the distance score is based on L2 norm Euclidean distance.

16. The computer-implemented method of clause 14, wherein, when the distance score is below a distance threshold, the trained CNV Caller Guide generates the output that specifies that the end of the CNV breakpoint is located on the subject candidate sequence.

17. The computer-implemented method of clause 16, wherein, when the distance score is above the distance threshold, the trained CNV Caller Guide generates the output that specifies that the end of the CNV breakpoint is not located on the subject candidate sequence.

18. The computer-implemented method of clause 7, wherein the anchor sequence and the candidate sequences each have 512 bases.

19. The computer-implemented method of clause 18, wherein the trained CNV Caller Guide processes one-hot encodings of the anchor sequence and the subject candidate sequence.

20. The computer-implemented method of clause 12, wherein the learned representation of the anchor sequence and the learned representation of the candidate sequences each have 64 dimensions.

21. The computer-implemented method of clause 7, wherein an in-training CNV Caller Guide is trained on a training dataset that comprises pairs of anchor and positive sequences that contain CNV breakpoint starts and CNV breakpoint ends, and negative sequences that do not contain the CNV breakpoint ends.

22. The computer-implemented method of clause 21, wherein anchor sequences are processed through an in-training encoder and an in-training multi-layer perceptron to generate learned representations of the anchor sequences, the positive sequences are processed through the in-training encoder and the in-training multi-layer perceptron to generate learned representations of the positive sequences, and the negative sequences are processed through the in-training encoder and the in-training multi-layer perceptron to generate learned representations of the negative sequences.

23. The computer-implemented method of clause 22, wherein the in-training CNV Caller Guide is trained to determine similarity between paired anchor and positive sequences and dissimilarity between the anchor sequences and the negative sequences by comparing the learned representations of the anchor sequences against the learned representations of the positive sequences and the negative sequences.

24. The computer-implemented method of clause 23, wherein the in-training CNV Caller Guide is trained using a triplet loss function that minimizes distances between paired anchor and positive sequences and maximizes distances between the anchor sequences and the negative sequences.

25. The computer-implemented method of clause 24, wherein the distances are measured based on L2 norm Euclidean distance.

26. The computer-implemented method of clause 24, wherein a triplet loss determined by the triplet loss function is zero when a distance between a pair of anchor and positive sequences plus a margin is less than a distance between the anchor sequence and a negative sequence.

27. The computer-implemented method of clause 26, wherein the triplet loss is non-zero when the distance between the pair of anchor and positive sequences plus the margin is more than the distance between the anchor sequence and the negative sequence.

28. The computer-implemented method of clause 27, wherein the margin is 2.

29. The computer-implemented method of clause 21, wherein the pairs of anchor and positive sequences include real pairs of anchor and positive sequences that contain observed CNV breakpoint starts and observed CNV breakpoint ends, and augmented pairs of anchor and positive sequences that contain harvested CNV breakpoint starts and harvested CNV breakpoint ends.

30. The computer-implemented method of clause 29, wherein the augmented pairs of anchor and positive sequences are harvested by sliding over the real pairs of anchor and positive sequences at upstream and downstream offsets from the observed CNV breakpoint starts and observed CNV breakpoint ends.

31. The computer-implemented method of clause 10, wherein the trained encoder is pre-trained as part of an autoencoder that is trained to encode a base sequence into a learned representation of the base sequence, and decode (reconstruct) the learned representation of the base sequence back into the base sequence.

32. The computer-implemented method of clause 31, wherein the autoencoder comprises groups of residual blocks, and skip connections,
wherein each group of residual blocks is parameterized by a number of convolution filters in residual blocks, a convolution window size of the residual blocks, and an atrous (dilated) convolution rate of the residual blocks,
wherein the convolution window size varies between the groups of residual blocks,
wherein the atrous convolution rate varies between the groups of residual blocks, and
wherein a training dataset for the autoencoder includes pairs of translated sequences generated from benign variants and pathogenic variants and used as benign training examples and as pathogenic training examples.

33. The computer-implemented method of clause 7, wherein the CNV Caller Guide is trained using semi-supervised learning.

34. The computer-implemented method of clause 32, wherein the autoencoder is trained using unsupervised learning.

35. The computer-implemented method of clause 16, wherein the distance threshold is determined based on a performance of the trained CNV Caller Guide on a validation set.

36. The computer-implemented method of clause 35, wherein the distance threshold is based on an average of a mean of distances determined by the trained CNV Caller Guide between pairs of anchor and positive sequences in the validation set and a mean of distances determined by the trained CNV Caller Guide between anchor sequences and negative sequences in the validation set.

37. The computer-implemented method of clause 36, wherein the distance threshold is based on an average of upper n percentiles of the distances determined by the trained CNV Caller Guide between the pairs of anchor and positive sequences in the validation set and lower n percentiles of the distances determined by the trained CNV Caller Guide between anchor sequences and negative sequences in the validation set.

38. The computer-implemented method of clause 37, wherein the n is 500.

39. A system, comprising:
a copy number variation (CNV) caller configured to:
   process (i) an anchor sequence with a start of a CNV breakpoint, and (ii) a candidate sequence; and
   generate an output that specifies whether the candidate sequence contains an end of the CNV breakpoint.

### Clause Set 2—Predicting CNV Start Breakpoint

1. A computer-implemented method, including:
processing sequence data that comprises:
   a start of a copy number variation (CNV) breakpoint,
   an end of the CNV breakpoint,
      wherein a location of the start of the CNV breakpoint in the sequence data is unknown, and
      wherein a location of the end of the CNV breakpoint in the sequence data is known; and
based on the processing, determining the location of the start of the CNV breakpoint in the sequence data.

2. The computer-implemented method of clause 1, wherein the sequence data has an anchor sequence.

3. The computer-implemented method of clause 2, wherein the sequence data has a plurality of candidate sequences.

4. The computer-implemented method of clause 3, wherein candidate sequences in the plurality of candidate sequences are upstream to the anchor sequence.

5. The computer-implemented method of clause 2, wherein the end of the CNV breakpoint is located on the anchor sequence.

6. The computer-implemented method of clause 3, wherein the start of the CNV breakpoint is located on one or more of the candidate sequences.

7. The computer-implemented method of clause 4, wherein a trained CNV Caller Guide processes the anchor sequence and a subject candidate sequence as inputs and generates an output that specifies whether the start of the CNV breakpoint is located on the subject candidate sequence.

8. The computer-implemented method of clause 7, wherein the output is one when the start of the CNV breakpoint is located on the subject candidate sequence.

9. The computer-implemented method of clause 8, wherein the output is zero when the start of the CNV breakpoint is not located on the subject candidate sequence.

10. The computer-implemented method of clause 7, wherein the trained CNV Caller Guide comprises a trained encoder and a trained multi-layer perceptron.

11. The computer-implemented method of clause 10, wherein the trained CNV Caller Guide processes the anchor sequence through the trained encoder and the trained multi-layer perceptron and generates a learned representation of the anchor sequence.

12. The computer-implemented method of clause 11, wherein the trained CNV Caller Guide processes the subject candidate sequence through the trained encoder and the trained multi-layer perceptron and generates a learned representation of the subject candidate sequence.

13. The computer-implemented method of clause 12, wherein the trained CNV Caller Guide determines a similarity (homology) between the anchor sequence and the subject candidate sequence by comparing the learned representation of the anchor sequence against the learned representation of the subject candidate sequence.

14. The computer-implemented method of clause 13, wherein the trained CNV Caller Guide measures the similarity using a distance score.

15. The computer-implemented method of clause 14, wherein the distance score is based on L2 norm Euclidean distance.

16. The computer-implemented method of clause 14, wherein, when the distance score is below a distance threshold, the trained CNV Caller Guide generates the output that specifies that the start of the CNV breakpoint is located on the subject candidate sequence.

17. The computer-implemented method of clause 16, wherein, when the distance score is above the distance threshold, the trained CNV Caller Guide generates the output that specifies that the start of the CNV breakpoint is not located on the subject candidate sequence.

18. The computer-implemented method of clause 7, wherein the anchor sequence and the candidate sequences each have 512 bases.

19. The computer-implemented method of clause 18, wherein the trained CNV Caller Guide processes one-hot encodings of the anchor sequence and the subject candidate sequence.

20. The computer-implemented method of clause 12, wherein the learned representations of the anchor sequences and the learned representations of the candidate sequences each have 64 dimensions.

21. The computer-implemented method of clause 7, wherein the in-training CNV Caller Guide is trained on a training dataset that comprises pairs of anchor and positive sequences that contain CNV breakpoint ends and starts, and negative sequences that do not contain the CNV breakpoint starts.

22. The computer-implemented method of clause 21, wherein the anchor sequences are processed through an in-training encoder and an in-training multi-layer perceptron to generate learned representations of the anchor sequences, the positive sequences are processed through the in-training encoder and the in-training multi-layer perceptron to generate learned representations of the positive sequences, and the negative sequences are processed through the in-training encoder and the in-training multi-layer perceptron to generate learned representations of the negative sequences.

23. The computer-implemented method of clause 22, wherein the in-training CNV Caller Guide is trained to determine similarity between paired anchor and positive sequences and dissimilarity between the anchor sequences and the negative sequences by comparing the learned representations of the anchor sequences against the learned representations of the positive sequences and the negative sequences.

24. The computer-implemented method of clause 23, wherein the in-training CNV Caller Guide is trained using a triplet loss function that minimizes distances between paired anchor and positive sequences and maximizes distances between the anchor sequences and the negative sequences.

25. The computer-implemented method of clause 24, wherein the distances are measured based on L2 norm Euclidean distance.

26. The computer-implemented method of clause 24, wherein a triplet loss determined by the triplet loss function is zero when a distance between a pair of anchor and positive sequences plus a margin is less than a distance between the anchor sequence and a negative sequence.

27. The computer-implemented method of clause 26, wherein the triplet loss is non-zero when the distance between the pair of anchor and positive sequences plus the margin is more than the distance between the anchor sequence and the negative sequence.

28. The computer-implemented method of clause 27, wherein the margin is 2.

29. The computer-implemented method of clause 21, wherein the pairs of anchor and positive sequences include real pairs of anchor and positive sequences that contain observed CNV breakpoint ends and starts, and augmented pairs of anchor and positive sequences that contain harvested CNV breakpoint ends and starts.

30. The computer-implemented method of clause 29, wherein the augmented pairs of anchor and positive sequences are harvested by sliding over the real pairs of anchor and positive sequences at upstream and downstream offsets from the observed CNV breakpoint ends and starts.

31. The computer-implemented method of clause 10, wherein the trained encoder is pre-trained as part of an autoencoder that is trained to encode a base sequence into a learned representation of the base sequence, and decode (reconstruct) the learned representation of the base sequence back into the base sequence.

32. The computer-implemented method of clause 31, wherein the autoencoder comprises groups of residual blocks, and skip connections,
wherein each group of residual blocks is parameterized by a number of convolution filters in the residual blocks, a convolution window size of the residual blocks, and an atrous (dilated) convolution rate of the residual blocks,
wherein the convolution window size varies between the groups of residual blocks,
wherein the atrous convolution rate varies between the groups of residual blocks, and
wherein a training dataset for the autoencoder includes pairs of translated sequences generated from benign variants and pathogenic variants and used as benign training examples and as pathogenic training examples.

33. The computer-implemented method of clause 7, wherein the CNV Caller Guide is trained using semi-supervised learning.

34. The computer-implemented method of clause 32, wherein the autoencoder is trained using unsupervised learning.

35. The computer-implemented method of clause 16, wherein the distance threshold is determined based on a performance of the trained CNV Caller Guide on a validation set.

36. The computer-implemented method of clause 35, wherein the distance threshold is based on an average of a mean of distances determined by the trained CNV Caller Guide between pairs of anchor and positive sequences in the validation set and a mean of distances determined by the trained CNV Caller Guide between the anchor sequences and negative sequences in the validation set.

37. The computer-implemented method of clause 36, wherein the distance threshold is based on an average of upper n percentiles of the distances determined by the trained CNV Caller Guide between the pairs of anchor and positive sequences in the validation set and lower n percentiles of the distances determined by the trained CNV Caller Guide between the anchor sequences and negative sequences in the validation set.

38. The computer-implemented method of clause 37, wherein the n is 500.

39. A system, comprising:
a copy number variation (CNV) caller configured to:
   process (i) an anchor sequence with an end of a CNV breakpoint, and (ii) a candidate sequence; and
   generate an output that specifies whether the candidate sequence contains a start of the CNV breakpoint.

### Clause Set 3—Predicting Both CNV Start and End Breakpoints

1. A computer-implemented method, including:
processing sequence data that comprises:
   a start of a copy number variation (CNV) breakpoint,
   an end of the CNV breakpoint,
      wherein a location of the start of the CNV breakpoint in the sequence data is unknown, and
      wherein a location of the end of the CNV breakpoint in the sequence data is unknown; and
based on the processing, determining
   the location of the start of the CNV breakpoint in the sequence data, and
   the location of the end of the CNV breakpoint in the sequence data.

2. A computer-implemented method, including:
processing sequence data that comprises:
   a start of a copy number variation (CNV) breakpoint,
   an end of the CNV breakpoint,
based on the processing, determining
   a location of the start of the CNV breakpoint in the sequence data, and/or
   a location of the end of the CNV breakpoint in the sequence data.

3. A system, comprising:
a copy number variation (CNV) caller configured to:
   process sequence data that comprises:
      a start of a CNV breakpoint,
      an end of the CNV breakpoint; and
   generate an output that specifies whether the sequence data contains the start of the CNV breakpoint and/or the end of the CNV breakpoint.

What we claim is:

1. A system comprising:
at least one processor; and
a non-transitory computer readable storage medium storing instructions that, when executed by the at least one processor, cause the system to:
process sequence data that comprises:
a start of a copy number variation (CNV) breakpoint,
an end of the CNV breakpoint,
wherein a location of the start of the CNV breakpoint in the sequence data is known, and
wherein a location of the end of the CNV breakpoint in the sequence data is unknown; and
based on the processing, determine the location of the end of the CNV breakpoint in the sequence data.

2. The system of claim 1, wherein the sequence data has an anchor sequence.

3. The system of claim 2, wherein the sequence data has a plurality of candidate sequences.

4. The system of claim 3, wherein candidate sequences in the plurality of candidate sequences are downstream to the anchor sequence.

5. The system of claim 2, wherein the start of the CNV breakpoint is located on the anchor sequence.

6. The system of claim 3, wherein the end of the CNV breakpoint is located on one or more of the plurality of candidate sequences.

7. The system of claim 3, further storing instructions that, when executed by the at least one processor, cause the system to:
process, by a trained CNV Caller Guide, the anchor sequence and a subject candidate sequence as inputs; and
generate, from the trained CNV Caller Guide, an output that specifies whether the end of the CNV breakpoint is located on the subject candidate sequence.

8. The system of claim 7, wherein the trained CNV Caller Guide comprises a trained encoder and a trained multi-layer perceptron.

9. The system of claim 8, further storing instructions that, when executed by the at least one processor, cause the system to:
process the anchor sequence through the trained encoder and the trained multi-layer perceptron of the trained CNV Caller Guide; and
generate, from the trained CNV Caller Guide, a learned representation of the anchor sequence.

10. A non-transitory computer readable storage medium storing instructions that, when executed by at least one processor, cause a system to:
process sequence data that comprises:
a start of a copy number variation (CNV) breakpoint,
an end of the CNV breakpoint,
wherein a location of the start of the CNV breakpoint in the sequence data is known, and
wherein a location of the end of the CNV breakpoint in the sequence data is unknown; and
based on the processing, determine the location of the end of the CNV breakpoint in the sequence data.

11. The non-transitory computer readable storage medium of claim 10, wherein the sequence data has an anchor sequence and a plurality of candidate sequences.

12. The non-transitory computer readable storage medium of claim 11, further storing instructions that, when executed by the at least one processor, cause the system to:
process the anchor sequence through a trained encoder and a trained multi-layer perceptron of a trained CNV Caller Guide; and
generate, from the trained CNV Caller Guide, a learned representation of the anchor sequence.

13. The non-transitory computer readable storage medium of claim 12, further storing instructions that, when executed by the at least one processor, cause the system to:
process a subject candidate sequence of the plurality of candidate sequences through a trained encoder and a trained multi-layer perceptron of the trained CNV Caller Guide; and
generate, from the trained CNV Caller Guide, a learned representation of the subject candidate sequence.

14. The non-transitory computer readable storage medium of claim 13, further storing instructions that, when executed by the at least one processor, cause the system to determine, by the trained CNV Caller Guide, a similarity between the anchor sequence and a subject candidate sequence of the plurality of candidate sequences by comparing the learned representation of the anchor sequence against the learned representation of the subject candidate sequence.

15. A computer-implemented method, including:
processing sequence data that comprises:
a start of a copy number variation (CNV) breakpoint,
an end of the CNV breakpoint,
wherein a location of the start of the CNV breakpoint in the sequence data is known, and
wherein a location of the end of the CNV breakpoint in the sequence data is unknown; and
based on the processing, determining the location of the end of the CNV breakpoint in the sequence data.

16. The computer-implemented method of claim 15, wherein a trained CNV Caller Guide processes the sequence data comprising an anchor sequence and a subject candidate sequence as inputs and generates an output that specifies whether the end of the CNV breakpoint is located on the subject candidate sequence.

17. The computer-implemented method of claim 16, wherein:
the trained CNV Caller Guide processes the anchor sequence through a trained encoder and a trained multi-layer perceptron and generates a learned representation of the anchor sequence; and
the trained CNV Caller Guide processes the subject candidate sequence through the trained encoder and the trained multi-layer perceptron and generates a learned representation of the subject candidate sequence.

18. The computer-implemented method of claim 17, wherein the trained CNV Caller Guide determines a similarity between the anchor sequence and the subject candidate sequence by comparing the learned representation of the anchor sequence against the learned representation of the subject candidate sequence.

19. The computer-implemented method of claim 18, wherein the trained CNV Caller Guide measures the similarity using a distance score.

20. The computer-implemented method of claim 19, wherein:
when the distance score is below a distance threshold, the trained CNV Caller Guide generates the output that specifies that the end of the CNV breakpoint is located on the subject candidate sequence; or
wherein, when the distance score is above the distance threshold, the trained CNV Caller Guide generates the output that specifies that the end of the CNV breakpoint is not located on the subject candidate sequence.
